SEQUENCE LISTING (59 sequences)

SEQ ID NO: 1 - DNA, 5097 bp, Homo sapiens
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgccccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtctgttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaatta

SEQ ID NO: 2 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 3 - DNA, 360 bp, Homo sapiens
ttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtttctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtg

SEQ ID NO: 4 - DNA, 30 bp, Artificial Sequence
  Description of Artificial Sequence Synthetic primer
nggtcctggttttaaattatggagtatgtt

SEQ ID NO: 5 - DNA, 30 bp, Artificial Sequence
  Description of Artificial Sequence Synthetic primer
nccaggaggttttaaattatggagtatgtg

SEQ ID NO: 6 - DNA, 26 bp, Artificial Sequence
  Description of Artificial Sequence Synthetic primer
gaaccagaatattctcgtctccacag

SEQ ID NO: 7 - DNA, 25 bp, Artificial Sequence
  Description of Artificial Sequence Synthetic primer
ctgtggagacgagaatattctggtt

SEQ ID NO: 8 - DNA, 25 bp, Artificial Sequence
  Description of Artificial Sequence Synthetic primer
atgatgagcaagctttctcacaagc

SEQ ID NO: 9 - DNA, 26 bp, Artificial Sequence
  Description of Artificial Sequence Synthetic primer
agcaagtatgatgagcaagctttctc

SEQ ID NO: 10 - DNA, 25 bp, Artificial Sequence
  Description of Artificial Sequence Synthetic primer
gcagcaagtatgatgagcaagcttt

SEQ ID NO: 11 - DNA, 23 bp, Artificial Sequence
  Description of Artificial Sequence Synthetic primer
gctttctcacaagcatttggttt

SEQ ID NO: 12 - DNA, 31 bp, Artificial Sequence
  Description of Artificial Sequence Synthetic primer
cacaagcatttggttttaaattatggagtat

SEQ ID NO: 13 - DNA, 22 bp, Artificial Sequence
  Description of Artificial Sequence Synthetic primer
atgatgagcaagctttctcaca

SEQ ID NO: 14 - DNA, 32 bp, Artificial Sequence
  Description of Artificial Sequence Synthetic primer
tctcacaagcatttggttttaaattatggagt

SEQ ID NO: 15 - DNA, 23 bp, Artificial Sequence
  Description of Artificial Sequence Synthetic primer
ntgtccactcgtctccacagaca

SEQ ID NO: 16 - DNA, 23 bp, Artificial Sequence
  Description of Artificial Sequence Synthetic primer
ntgtccactcgtctccacagaca

SEQ ID NO: 17 - DNA, 16 bp, Artificial Sequence
  Description of Artificial Sequence Synthetic primer
ctcgtctccacagaca

SEQ ID NO: 18 - DNA, 22 bp, Artificial Sequence
  Description of Artificial Sequence Synthetic primer
ntgtccactcgtctccacagac

SEQ ID NO: 19 - DNA, 22 bp, Artificial Sequence
  Description of Artificial Sequence Synthetic primer
ntgtccactcgtctccacagac

SEQ ID NO: 20 - DNA, 15 bp, Artificial Sequence
  Description of Artificial Sequence Synthetic primer
ctcgtctccacagac

SEQ ID NO: 21 - DNA, 21 bp, Artificial Sequence
  Description of Artificial Sequence Synthetic primer
ncactctcgtctccacaggca

SEQ ID NO: 22 - DNA, 21 bp, Artificial Sequence
  Description of Artificial Sequence Synthetic primer
ncactctcgtctccacaggca

SEQ ID NO: 23 - DNA, 18 bp, Artificial Sequence
  Description of Artificial Sequence Synthetic primer
ctctcgtctccacaggca

SEQ ID NO: 24 - DNA, 21 bp, Artificial Sequence
  Description of Artificial Sequence Synthetic primer
ncactctcgtctccacggaca

SEQ ID NO: 25 - DNA, 21 bp, Artificial Sequence
  Description of Artificial Sequence Synthetic primer
ncactctcgtctccacggaca

SEQ ID NO: 26 - DNA, 18 bp, Artificial Sequence
  Description of Artificial Sequence Synthetic primer
ctctcgtctccacggaca

SEQ ID NO: 27 - DNA, 23 bp, Artificial Sequence
  Description of Artificial Sequence Synthetic primer
nacaggtctcgtctccacagaaa

SEQ ID NO: 28 - DNA, 23 bp, Artificial Sequence
  Description of Artificial Sequence Synthetic primer
nacaggtctcgtctccacagaaa

SEQ ID NO: 29 - DNA, 16 bp, Artificial Sequence
  Description of Artificial Sequence Synthetic primer
ctcgtctccacagaaa

SEQ ID NO: 30 - DNA, 22 bp, Artificial Sequence
  Description of Artificial Sequence Synthetic primer
nacaggtctcgtctccacagaa

SEQ ID NO: 31 - DNA, 22 bp, Artificial Sequence
  Description of Artificial Sequence Synthetic primer
nacaggtctcgtctccacagaa

SEQ ID NO: 32 - DNA, 15 bp, Artificial Sequence
  Description of Artificial Sequence Synthetic primer
ctcgtctccacagaa

SEQ ID NO: 33 - DNA, 22 bp, Artificial Sequence
  Description of Artificial Sequence Synthetic primer
nggtctctcgtctccacaggaa

SEQ ID NO: 34 - DNA, 22 bp, Artificial Sequence
  Description of Artificial Sequence Synthetic primer
nggtctctcgtctccacaggaa

SEQ ID NO: 35 - DNA, 18 bp, Artificial Sequence
  Description of Artificial Sequence Synthetic primer
ctctcgtctccacaggaa

SEQ ID NO: 36 - DNA, 23 bp, Artificial Sequence
  Description of Artificial Sequence Synthetic primer
nggtactctcgtctccacggaaa

SEQ ID NO: 37 - DNA, 23 bp, Artificial Sequence
  Description of Artificial Sequence Synthetic primer
nggtactctcgtctccacggaaa

SEQ ID NO: 38 - DNA, 19 bp, Artificial Sequence
  Description of Artificial Sequence Synthetic primer
actctcgtctccacggaaa

SEQ ID NO: 39 - DNA, 29 bp, Artificial Sequence
  Description of Artificial Sequence Synthetic primer
nacaggtttttaaattatggagtatgtgt

SEQ ID NO: 40 - DNA, 29 bp, Artificial Sequence
  Description of Artificial Sequence Synthetic primer
nacaggtttttaaattatggagtatgtgt

SEQ ID NO: 41 - DNA, 22 bp, Artificial Sequence
  Description of Artificial Sequence Synthetic primer
ttttaaattatggagtatgtgt

SEQ ID NO: 42 - DNA, 29 bp, Artificial Sequence
  Description of Artificial Sequence Synthetic primer
nacaggtgttttaaattatggagtatgtg

SEQ ID NO: 43 - DNA, 29 bp, Artificial Sequence
  Description of Artificial Sequence Synthetic primer
nacaggtgttttaaattatggagtatgtg

SEQ ID NO: 44 - DNA, 22 bp, Artificial Sequence
  Description of Artificial Sequence Synthetic primer
gttttaaattatggagtatgtg

SEQ ID NO: 45 - DNA, 30 bp, Artificial Sequence
  Description of Artificial Sequence Synthetic primer
ntgtccagttttaaattatggagtatgttt

SEQ ID NO: 46 - DNA, 30 bp, Artificial Sequence; identical to SEQ ID NO: 45
  Description of Artificial Sequence Synthetic primer

SEQ ID NO: 47 - DNA, 23 bp, Artificial Sequence
  Description of Artificial Sequence Synthetic primer
gttttaaattatggagtatgttt

SEQ ID NO: 48 - DNA, 29 bp, Artificial Sequence
  Description of Artificial Sequence Synthetic primer
ntgtccagttttaaattatggagtatgtt

SEQ ID NO: 49 - DNA, 29 bp, Artificial Sequence
  Description of Artificial Sequence Synthetic primer
ntgtccagttttaaattatggagtatgtt

SEQ ID NO: 50 - DNA, 22 bp, Artificial Sequence
  Description of Artificial Sequence Synthetic primer
gttttaaattatggagtatgtt

SEQ ID NO: 51 - DNA, 26 bp, Artificial Sequence
  Description of Artificial Sequence Synthetic primer
gcctgtagttttacttactctcgtct

SEQ ID NO: 52 - DNA, 24 bp, Artificial Sequence
  Description of Artificial Sequence Synthetic primer
agcctgtagttttacttactctcg

SEQ ID NO: 53 - DNA, 20 bp, Artificial Sequence
  Description of Artificial Sequence Synthetic primer
agcattagaaagcctgtagt

SEQ ID NO: 54 - DNA, 26 bp, Artificial Sequence
  Description of Artificial Sequence Synthetic primer
gtagttttacttactctcgtctccac

SEQ ID NO: 55 - DNA, 30 bp, Artificial Sequence
  Description of Artificial Sequence Synthetic primer
tgtagttttacttactctcgtctccacaga

SEQ ID NO: 56 - DNA, 30 bp, Artificial Sequence; identical to SEQ ID NO: 5
  Description of Artificial Sequence Synthetic primer

SEQ ID NO: 57 - DNA, 23 bp, Artificial Sequence
  Description of Artificial Sequence Synthetic primer
ggttttaaattatggagtatgtg

SEQ ID NO: 58 - DNA, 30 bp, Artificial Sequence; identical to SEQ ID NO: 4
  Description of Artificial Sequence Synthetic primer

SEQ ID NO: 59 - DNA, 24 bp, Artificial Sequence
  Description of Artificial Sequence Synthetic primer
tggttttaaattatggagtatgtt

(19) United States
(12) Patent Application Publication
(10) Pub. No.: US 2008/0153097 A1
(43) Pub. Date: Jun. 26, 2008

(54) METHODS AND KITS FOR DETECTING JAK2 NUCLEIC ACID

(75) Inventors: Scott C. Johnson, Sun Prairie, WI (US); Kelly M. Homb, Monroe, WI (US); David J. Marshall, Madison, WI (US); Elizabeth K. Mulligan, Madison, WI (US)

Correspondence Address: FOLEY & LARDNER LLP, 150 EAST GILMAN STREET, P.O. BOX 1497, MADISON, WI, 53701-1497, US

(73) Assignee: EraGen Biosciences, Inc.

(21) Appl. No.: 11/940,875

(22) Filed: Nov. 15, 2007

Related U.S. Application Data
(60) Provisional application No. 60/859,185, filed on Nov. 15, 2006.

(51) Int. Cl. C12Q 1/68

(52) U.S. Cl. 435/6

(57) ABSTRACT

Disclosed are methods, kits, and components for detecting JAK2 nucleic acids in a sample. In one aspect, the methods may be used to detect mutant JAK2 nucleic acid in a mixture of mutant JAK2 nucleic acid and wild-type JAK2 nucleic acid. The methods utilize primers and reporter molecules comprising non-natural bases. The disclosed kits may include one or more components for performing the disclosed methods.

### CROSS-REFERENCE TO RELATED APPLICATIONS

The application claims priority to U.S. Provisional Application No. 60/859,185, filed Nov. 15, 2006, the entire contents of which are herein incorporated by reference.

### FIELD OF THE INVENTION

The present methods and kits relate broadly to the identification of organisms using nucleic acid amplification techniques. In particular, the methods and kits relate to methods of detecting Janus kinase 2 (“JAK2”) nucleic acid.

### BACKGROUND OF THE INVENTION

Human myeloproliferative disorders (MPDs) include a variety of malignant blood-cell diseases which are characterized by increased hematopoiesis leading to elevated numbers of nonlymphoid cells or platelets in the peripheral blood. These include polycythaemia vera (PV), essential thrombocythaemia (ET), and idiopathic myelofibrosis (IMF). Although the molecular pathogenesis of MPDs is unknown, based on the model of chronic myeloid leukaemia, it is expected that a constitutive tyrosine kinase activity could be at the origin of these diseases. Tyrosine kinases such as Janus kinase 2 (JAK2) have been implicated in several related disorders.

The JAK family of proteins mediate the effects of hematopoietic cytokines, for example, erythropoietin and granulocyte colony-stimulating factor (G-CSF), and by phosphorylating cytoplasmic targets, including signal transducers and activators of transcription (STATs). A mutation in the JAK2 protein that results in a phenylalanine for valine substitution at amino acid 617 (i.e., “Val617Phe” or “V617F”) has been identified in patients with MPDs. The JAK2 V617F mutant is a constitutively active tyrosine kinase that activates the STAT, mitogen activated protein kinase (MAPK) and phosphotidylinositol 3-kinase (PI3K) signalling pathways, and transforms haematopoietic progenitors. Acquisition of the (V617F) mutation within a multipotent progenitor is thought to be associated with a clonal proliferation of cells within the erythroid and myeloid lineages. Most patients appear to be heterozygous for the mutation however some patients appear homozygous as the result of mitotic recombination. The V617F substitution in the negative regulatory JH2 domain of JAK2 is predicted to deregulate kinase activity.

### SUMMARY OF THE INVENTION

There are provided herein methods and kits for quickly, easily and inexpensively detecting and distinguishing nucleic acids such as JAK2 nucleic acid, (e.g., wild-type (wt) JAK2 nucleic acid and/or a mutant JAK2 nucleic acid such as V617F). Thus in accordance with one aspect, the present invention provides methods of detecting wt JAK2 nucleic acid and mutant JAK2 nucleic acid in a sample, if present, comprising: (a) contacting the sample with: (i) a first primer suitable for amplifying a wt JAK2 nucleic acid, wherein the first primer comprises a first label and a first non-natural base; (ii) a second primer suitable for amplifying a mutant JAK2 nucleic acid, wherein the second primer comprises a second label and a second occurrence of the first non-natural base; (iii) a third primer suitable for amplifying both wt JAK2 nucleic acid and mutant JAK2 nucleic acid; and (iv) a reporter comprising a third label and a second non-natural base that base-pairs with the first non-natural base; (b) performing an amplification reaction comprising the primers of step (a) under conditions suitable to produce an amplification product of the wt JAK2 nucleic acid and the mutant JAK2 nucleic acid in the sample, if present, wherein the reporter is incorporated into the amplification products; and (c) detecting the amplification products produced in step (b), thereby determining the presence or absence of wt JAK2 nucleic acid, mutant JAK2 nucleic acid, or both in the sample. In one embodiment, the detection is accomplished by observing a signal from the first label, the second label, or both.

The methods may be used to specifically amplify wt JAK2 nucleic and/or mutant JAK2 nucleic acids. The amplification product of wt JAK2 nucleic acid typically incorporates a different specific primer from the amplification product of mutant JAK2 nucleic acid. The specific primer may include a first or second label and a first non-natural base and the methods may include incorporating in the amplification a labeled reporter which comprises a third label and a second non-natural base that base-pairs with the first non-natural base. In one embodiment, the amplification product may be detected by observing energy transfer (e.g., fluorescence energy transfer) or quenching between the first label and the third label. The methods may further include detecting the amplification products and distinguishing among wt JAK2 nucleic and mutant JAK2 nucleic based on the amplification products that are detected. In some embodiments, the methods include determining a melting temperature of the amplified JAK2 nucleic acid.

Typically, two specific primers are added to the sample, but the methods and kits are not so limited. The specific primers are non-identical in sequence, but can differ by only a single base. The two or more specific primers may comprise a label that is detectable, such as a fluorophore. Suitable fluorophores include, e.g., fluorescein and hexachlorofluorescein. Each specific primer can include a non-natural nucleotide base such as, but not limited to isocytosine or isoguanosine. The labels on the two or more specific primers may be the same or different. In suitable embodiments, the first and second labels are different. In other embodiments, all of the labels are different.

Inventive methods can further include adding a non-natural nucleotide base to the sample. Suitable non-natural nucleotide bases include, but are not limited to, isoguanosine or isocytosine. Typically the non-natural nucleotide base is complementary to the non-natural nucleotide base used in the specific primers. The non-natural nucleotide base can include a label, e.g., a fluorescence quencher such as dabcyl.

The amplification of JAK2 nucleic acid (e.g., wt JAK2 nucleic and/or mutant JAK2 nucleic acid) may be carried out with a nucleic acid polymerase using the polymerase chain reaction. Typically, the JAK2 nucleic acid is DNA (e.g., genomic or cDNA). In the course of the amplification, the non-natural nucleotide base is incorporated into amplification products. The amplification product of wt JAK2 nucleic acid incorporates a specific primer for JAK2 nucleic acid and the non-natural nucleotide base to produce a detectable change in a signal. Likewise, the amplification product of mutant JAK2 nucleic acid incorporates a specific primer for the mutant JAK2 nucleic acid and the non-natural nucleotide base to produce a detectable change in a signal. The signal change can be produced by any appropriate method known to those of skill in the art. For example, the signal change may be an increase or decrease in fluorescence. Moreover, the detection of the amplification products can occur during the amplification step (in real-time and/or continuously) or after the amplification step. A signal change may be observed by melting the amplification products.

Inventive methods may be employed for detecting a wide variety of JAK2 nucleic acids including wt human JAK2 nucleic acid (SEQ ID NO:1) and/or mutant JAK2 nucleic acids. In one embodiment, the mutant JAK2 nucleic acid is a V617F mutant nucleic acid (a mutant of SEQ ID NO:1 having a G2343T transversion). In another embodiment, the mutant JAK2 nucleic acid is a K607N mutant nucleic acid (a mutant of SEQ ID NO:1 having a G1821C transversion) or a fragment thereof. In another embodiment, the mutant JAK2 nucleic acid is a mutant having a F537-K539del-insL mutation (a mutant of SEQ ID NO:1 having a deletion at positions 1611-1616). In another embodiment, the mutant JAK2 nucleic acid is a mutant having a CAA to ATT mutation at positions 1614 through 1616 of SEQ ID NO:1, resulting in a H538Q and K539L mutation. In another embodiment, the mutant JAK2 nucleic

In some embodiments, the methods disclosed herein are used to detect JAK2 nucleic acid in a sample. The methods for detecting JAK2 nucleic acid in a sample may include: (a) reacting a mixture that includes (i) nucleic acid isolated from the sample; (ii) at least a first pair of specific primers (i.e., a forward primer and a reverse primer) capable of being used to amplify specifically JAK2 nucleic acid (e.g., wt or mutant JAK2 nucleic acid). Optionally, the mixture may include (iii) at least a second pair of specific primers (i.e., a forward primer and a reverse primer) capable of being used to amplify specifically JAK2 nucleic acid (e.g., wt or mutant JAK2 nucleic acid).

In some embodiments, the reaction mixture includes two pairs of primers for detecting wt and mutant JAK2 nucleic acid. For example, the reaction mixture may include two forward primers and two reverse primers for detecting wt and mutant JAK2 nucleic acid. In other embodiments, the reaction mixture may include two forward primers and a single reverse primer (or alternatively a single forward primer and two reverse primers) for detecting wt and mutant JAK2 nucleic acid. In other words, a single forward primer or a single reverse primer may be capable of specifically hybridizing to both wt and mutant JAK2 nucleic. In some embodiments, the reaction mixture may include a universal primer capable of amplifying both wt JAK2 nucleic acid and mutant JAK2 nucleic acid.

The methods disclosed herein may be used to detect mutant JAK2 nucleic acid in a mixture of mutant JAK2 nucleic acid and wt JAK2 nucleic acid. The mixture may include 1% or less mutant JAK2 nucleic acid relative to wt JAK2 nucleic acid, based on copy number. In some embodiments, the mixture may include 0.1% or less mutant JAK2 nucleic acid relative to wt JAK2 nucleic acid, based on copy number. The methods may be used to detect as few as 5 copies of JAK2 nucleic acid in a sample (e.g., as few as 5 copies of wt and/or mutant JAK2 nucleic acid in a sample).

The specific primers may be designed to have exact complementarity to the target JAK2 nucleic acid sequence or the specific primers may include mismatches. For example, the specific primers may include at least one non-natural nucleotide that does not base-pair with any corresponding nucleotide in the target nucleic acid sequence. In some embodiments, at least one of the first specific primer and second specific primer include a non-complementary tail at one end of the primer that does not base-pair with the target JAK2 nucleic acid sequence (e.g., the 5′ terminal nucleotide, which may include a non-standard base such as isocytosine or isoguanine). Likewise, the primers may have exact complementarity to wt JAK2 nucleic acid (e.g., complementarity to SEQ ID NO:1) or may include one or more mismatches with respect to the complement of wt JAK nucleic acid. In one embodiment, the primers may have exact complementarity to a mutant JAK2 nucleic acid.

In some embodiments, the reaction mixture includes two specific forward primers and/or two specific reverse primers. Where the reaction mixture include two specific forward primers, the two specific forward primers differ in sequence by at least one nucleotide (e.g., the 3′ terminal nucleotide or a nucleotide within 5 nucleotides from the 3′ terminal end). The reaction mixture may include two specific reverse primers, and optionally, the two specific reverse primers may differ in sequence by at least one nucleotide (e.g., the 3′ terminal nucleotide or a nucleotide within 5 nucleotides from the 3′ terminal end). For example, the reaction mixture may include a first specific forward primer that is specific for wt JAK2 nucleic acid and a second specific forward primer that is specific for mutant JAK2 nucleic acid. Likewise, the reaction mixture may include a first specific reverse primer that is specific for wt JAK2 nucleic acid and a second specific reverse primer that is specific for mutant JAK2 nucleic acid. Where two specific forward primers or two specific reverse primers are used, the reaction mixture may include a third primer, which is specific for both wt JAK2 nucleic acid and mutant JAK2 nucleic acid, i.e., a “universal” primer.

In some embodiments, at least one of the primer pair used to amplify the target nucleic acid comprises a label. Both members of the primer pair may comprise a label, which may be the same or different. In some embodiments, the reaction mixture includes a first forward primer (or first reverse primer) that is specific for a first target nucleic acid and comprises a first label. Optionally, the reaction mixture includes a second forward primer (or second reverse primer) that is specific for a second target nucleic acid and comprises a second label. The first label and the second label may be the same or different. In suitable embodiments, both the first specific primer and second specific primer comprise labels which are different. Suitable labels may include fluorophores and quenchers.

In some embodiments, at least one of the first specific primer and second specific primer may comprise a non-natural nucleotide base. In suitable embodiments, both the first specific primer and second specific primer may comprise a non-natural nucleotide base, which may be the same or different. Non-natural nucleotides may include nucleobases that do not base pair efficiently with A, C, G, T, or U under standard reaction conditions for performing PCR. Non-natural nucleotides may include isoguanosine or isocytidine (i.e., having guanine and cytosine as nucleobases).

The reaction mixture may include a reporter molecule. For example, the reaction mixture may include a labeled non-natural nucleotide as a reporter molecule. The labeled non-natural nucleotide may be capable of base-pairing with a corresponding non-natural nucleotide present in at least one of the primers of the reaction mixture (e.g., a first specific primer and/or a second specific primer, which may be forward and/or reverse primers). Suitable labels may include fluorophores and quenchers. In suitable embodiments, the non-natural nucleotide is labeled with a quencher that is capable of quenching a fluorophore that is used to label at least one of the first specific primer and the second specific primer (or different fluorophores that are used to label the first specific primer and the second specific primer). The non-natural nucleotide base may include isoguanosine or isocytidine. The non-natural nucleotide base may be present in a reaction mixture as a non-natural nucleotide triphosphate and may be incorporated into amplified nucleic acid (e.g., amplified wt JAK2 nucleic acid and/or mutant JAK2 nucleic acid) by a nucleic acid polymerase.

In the present methods, detecting the wt and/or mutant JAK2 nucleic acid may include observing a change in a signal of a label present in at least one primer of the reaction mixture (e.g., a first specific primer and/or a second specific primer, which may be forward and/or reverse primers). In some embodiments, detecting may include observing a change in a signal in a label that is present in a reporter present in the reaction mixture. Detecting may include observing a change in a signal from a label of a primer and a label of a reporter. Detecting a change in a signal may include detecting a change in fluorescence, such as observing a decrease in fluorescence, observing an increase in fluorescence, observing fluorescence polarization, and observing fluorescence depolarization. Detecting may include determining a melting temperature of amplified nucleic acid.

The primers of the methods may be used to amplify any suitable target nucleic acid. In some embodiments, at least one primer of the reaction mixture is used to amplify JAK2 nucleic acid, which may include wt JAK2 nucleic acid and/or mutant JAK2 nucleic acid (e.g., JAK2 V617F mutant nucleic acid). Optionally, the primers of the methods may be used to amplify a control nucleic acid (e.g., the reaction mixture may include primers for amplifying a control nucleic acid which is present in the sample or which is added to the sample).

Certain embodiments of the methods described herein are suitable for detecting a wt or mutant JAK2 nucleic acid in a mixed population of JAK2 nucleic acids (e.g., polymorphic JAK2 nucleic acid that includes a mixture of wt JAK2 nucleic acid and one or more mutant JAK2 nucleic acids). For example, the methods may be used to detect mutant JAK2 nucleic acid when the mutant JAK2 nucleic acid represents no more than about 2% of the total population of JAK2 nucleic acid in a mixed population. The method may be useful for detecting mutant JAK2 nucleic acid when the mutant JAK2 nucleic acid represents no more than about 2%, no more than about 1% or no more than about 0.1% of the total population of JAK2 nucleic acid in a sample.

Also disclosed are polynucleotides useful for detecting JAK2 nucleic acid and/or mutant JAK2 nucleic acid. For example, in the present methods, the reaction mixture may include any of SEQ ID NOS: 5, 15-26, 39-44, 56-57, or complements thereof as a first specific primer. The reaction mixture may include any of SEQ ID NOS: 4, 27-38, 45-50, 58-59, or complements thereof as a second specific primer. The reaction mixture may include any of SEQ ID NOS: 6-14, 51-55, or complements thereof as a third specific primer. In some embodiments of methods disclosed herein, the first primer comprises SEQ ID NO: 5, the second primer comprises SEQ ID NO: 4, and the third primer comprises SEQ ID NO: 6. In another embodiment, the first primer comprises SEQ ID NO:39, the second primer comprises SEQ ID NO:45, and the third primer comprises SEQ ID NO:52. In yet another embodiment, the first primer comprises SEQ ID NO:21, the second primer comprises SEQ ID NO:36, and the third primer is selected from the group consisting of: SEQ ID NO: 9, 10, and 12.

Also disclosed are polynucleotides having significant sequence identity to the polynucleotide sequence of SEQ ID NOS: 4-59, or complements thereof. For example, polynucleotides having at least about 95% sequence identity (or at least about 96%, 97%, 98%, or 99% sequence identity) are contemplated, where the polynucleotide having 95% sequence identity (or 96%, 97%, 98%, or 99% sequence identity) can function as a primer for a respective target nucleic acid (e.g., wt JAK2 nucleic acid and/or mutant JAK2 nucleic acid). Variant polynucleotides as envisioned herein may include polynucleotides that differ from any one of SEQ ID NOS: 4-59 by one, two, three, four, or five bases, so long as the polynucleotide is capable of specifically hybridizing to the respective target nucleic acid under stringent hybridization conditions. In particular embodiments, the polynucleotides are capable of specifically hybridizing to the respective target and are capable of being extended in an amplification reaction. Variant polynucleotides may also have a 5 nucleotide sequence at the 3′ terminus that differs from a 5 nucleotide sequence at the 3′ terminus of any one of SEQ ID NOS: 4-59 by a single nucleotide (e.g., the 3′ terminal nucleotide). Variant polynucleotides may further comprise a 5′ tail sequence of about 1-5, 1-10, 2-10, 3-10, 4-10, 5-10 or more nucleotides in length, which is not capable of specifically hybridizing to the target nucleic acid. The tail sequence may comprise one or more non-standard bases.

In some embodiments, the reaction mixture further includes an amplification mixture. The reaction mixture may include one or more of nucleotides (e.g., dATP, dCTP, dGTP, dTTP, UTP), salts, buffers, surfactants, enzymes, and the like. The reaction mixture may include nucleotide analogs such as nucleotides with thio-substituted phosphates (e.g., a thio analog of at least one of dATP, dCTP, dGTP, dTTP, UTP, or non-natural nucleotides such as deoxy iso-cytidine triphosphate (diCTP) and deoxy iso-guano sine triphosphate (diGTP)) that includes a sulfur atom instead of an oxygen atom in the alpha, beta, or gamma position of the triphosphate). The reaction mixture may include dideoxy analogs of nucleotides (i.e., a 2′,3′-dideoxy analog of at least one of ATP, CTP, GTP, TTP, UTP, or non-natural nucleotides such as iCTP and iGTP). The reaction mixture may include phosphoramidite analogs of nucleotides (e.g., a 3′ phosphoramidite analog of at least one of dATP, dCTP, dGTP, dTTP, and UTP or non-natural nucleotides such as diCTP and diGTP).

In another aspect of the methods disclosed herein, there are provided kits for detecting and/or distinguishing JAK2 nucleic acid in a sample according to the methods disclosed herein. The kits may include: (1) a first specific primer pair for amplifying a mutant JAK2 nucleic acid, wherein at least one member of the first primer pair comprises a label and a non-standard base; (2) optionally, a second specific primer pair for amplifying a wt JAK2 nucleic acid, wherein at least one member of the second primer pair comprises a label and a non-standard base; (3) optionally, a third specific primer pair for amplifying a control nucleic acid; and (4) a reporter comprising a non-natural nucleotide base. The first primer pair and the second primer pair may have a primer in common (e.g., a universal forward or reverse primer). The first, second, and third specific primers may include a first, second, and third non-natural nucleotide base complementary to the non-natural nucleotide base of the reporter included in the kit. The non-natural nucleotide base of the primers may be the same or different. In one embodiment, the first, second, and/or third specific primer includes iso-C and the non-natural nucleotide base of the reporter includes iso-G. In some such embodiments, the first specific primer comprises the nucleotide sequence of SEQ ID NO:17, 20, 23, 26, 41, 44, or 57; the second specific primer comprises the nucleotide sequence of SEQ ID NO:29, 32, 35, 38, 47, 50, 57, or 59; and the non-natural nucleotide base comprises iso-C or iso-G. In other such embodiments, the first, second, and/or third specific primers and the non-natural nucleotide base each independently comprise a label which may be the same or different. For example, the labels of the first, second, and/or third specific primers can be fluorophores (which may be the same or different) and the label of the non-natural nucleotide base can be a fluorescence quencher that is capable of quenching one or more fluorophores of the primers. Optionally, the kit further comprises other components such as buffers and reagents to perform the methods disclosed herein.

Also disclosed are kits that include at least one component for performing the methods disclosed herein. For example, a kit may include at least one component for detecting JAK2 nucleic acid in a sample. For example, a kit may include (i) a first specific primer capable of specifically amplifying mutant JAK2 nucleic acid (e.g., SEQ ID NO:1); (ii) optionally, a second specific primer capable of specifically amplifying wt JAK2 nucleic acid (e.g., SEQ ID NO:3); and (iii) optionally, a third specific primer capable of amplifying a control nucleic acid. In some embodiments, at least one of the first, second, and third specific primer may include a non-natural nucleotide base. Typically, at least one of the first, second, and third specific primer comprises a label, which may be the same or different. Suitable labels may include fluorophores and quenchers. The kit also may include a reporter molecule. For example, the kit may include a non-natural nucleotide coupled (e.g., covalently conjugated) to a quencher that is capable of quenching a fluorophore present in at least one of the first, second, and third specific primer.

The kit may include a first primer having the polynucleotide sequence of SEQ ID NO:5 or having a sequence with substantial polynucleotide sequence identity to SEQ ID NO:5 (e.g., a sequence having at least about 95%, 96%, 97%, 98%, or 99% sequence identity to SEQ ID NO:5). A first primer having substantial identity to SEQ ID NO:5 may be used to amplify a respective target (e.g., wt JAK2 nucleic acid). The kit may include a second primer having the sequence of SEQ ID NO:4 or having a sequence with substantial identity to SEQ ID NO:4 (e.g., a sequence having at least about 95%, 96%, 97%, 98%, or 99% sequence identity to SEQ ID NO:4). A second primer having substantial identity to SEQ ID NO:4 may be used to amplify a respective target (e.g., mutant JAK2 nucleic acid). The kit may include a universal primer capable of amplifying both wt JAK2 nucleic acid and mutant JAK2 nucleic acid (e.g., a primer comprising SEQ ID NO:6 or SEQ ID NO:7).

The kit may include a first primer having the polynucleotide sequence of SEQ ID NO:39 or having a sequence with substantial polynucleotide sequence identity to SEQ ID NO:39 (e.g., a sequence having at least about 95%, 96%, 97%, 98%, or 99% sequence identity to SEQ ID NO:39). A first primer having substantial identity to SEQ ID NO:39 may be used to amplify a respective target (e.g., wt JAK2 nucleic acid). The kit may include a second primer having the sequence of SEQ ID NO:45 or having a sequence with substantial identity to SEQ ID NO:45 (e.g., a sequence having at least about 95%, 96%, 97%, 98%, or 99% sequence identity to SEQ ID NO:45). A second primer having substantial identity to SEQ ID NO:45 may be used to amplify a respective target (e.g., mutant JAK2 nucleic acid). The kit may include a universal primer capable of amplifying both wt JAK2 nucleic acid and mutant JAK2 nucleic acid (e.g., a primer comprising SEQ ID NO:52).

The kit may include a first primer having the polynucleotide sequence of SEQ ID NO:21 or having a sequence with substantial polynucleotide sequence identity to SEQ ID NO:21 (e.g., a sequence having at least about 95%, 96%, 97%, 98%, or 99% sequence identity to SEQ ID NO:21). A first primer having substantial identity to SEQ ID NO:21 may be used to amplify a respective target (e.g., wt JAK2 nucleic acid). The kit may include a second primer having the sequence of SEQ ID NO:36 or having a sequence with substantial identity to SEQ ID NO:36 (e.g., a sequence having at least about 95%, 96%, 97%, 98%, or 99% sequence identity to SEQ ID NO:36). A second primer having substantial identity to SEQ ID NO:36 may be used to amplify a respective target (e.g., mutant JAK2 nucleic acid). The kit may include a universal primer capable of amplifying both wt JAK2 nucleic acid and mutant JAK2 nucleic acid (e.g., a primer comprising SEQ ID NO:9, 10, or 12).

The kit may include additional components. For example, the kit may include components to provide an amplification mixture.

Also disclosed herein are polynucleotides. For example, polynucleotides as disclosed herein may include a polynucleotide, optionally coupled (e.g., covalently conjugated) to a label, where the polynucleotide has at least about 95% sequence identity (or 96%, 97%, 98%, or 99% sequence identity) to a polynucleotide selected from SEQ ID NOs:4-59. Typically, the polynucleotide is capable of being used as a primer for amplifying JAK2 nucleic acid. Polynucleotides as disclosed herein may also include polynucleotides that hybridize under stringent conditions to a polynucleotide selected from SEQ ID NOs:4-59 or to the complement of a polynucleotide selected from SEQ ID NOs:4-59. Polynucleotides, as disclosed herein, may include a polynucleotide selected from SEQ ID NOs:4-59 in which at least one nucleotide has been replaced with a nucleotide having a non-natural base (e.g., isocytidine and isoguanosine). Variant polynucleotides as envisioned herein may include polynucleotides that differs from any one of SEQ ID NO: 4-59 by one, two, three, four, or five bases, so long as the polynucleotide is capable of specifically hybridizing to the respective target nucleic acid under stringent hybridization conditions. In particular embodiments, the polynucleotides are capable of specifically hybridizing to the respective target and are capable of being extending in an amplification reaction. Variant polynucleotides may also have a 5 nucleotide sequence at the 3′ terminus that differs from a 5 nucleotide sequence at the 3′ terminus of any one of SEQ ID NOS: 4-59 by a single nucleotide. Polynucleotides, as disclosed herein, may include polynucleotides having a 5-nucleotide sequence at the 3′ terminus that differs from a 5-nucleotide sequence of any one of SEQ ID NOs:4-59 by a single nucleotide (e.g., a single nucleotide at the 3′ end), and otherwise the polynucleotide may be at least about 95% identical to any one of SEQ ID NOs:4-59. Polynucleotides, as disclosed herein, may include polynucleotides that differ by a single 3′ terminal nucleotide with respect to SEQ ID NOs:4-59.

The methods and kits can be applied to a wide variety of detection technologies including “real time” or “continuous” detection technologies. In addition, the methods and kits disclosed herein can be incorporated into a variety of mass screening techniques and readout platforms (e.g., microarrays). The methods may be performed in solution. In some embodiments, the methods are performed with a solid substrate to which at least one component of the method or kit is immobilized. For example, the component may be covalently immobilized or non-covalently immobilized to the solid substrate.

### BRIEF DESCRIPTION OF THE DRAWINGS

FIG. 1 illustrates concentration standard reproducibility of the MultiCode-RTx JAK2 V617F assay on two different Light Cycler instruments (FIGS. 1A and 1B). The graphs show log ΔCt versus DNA concentration.

FIG. 2 illustrates analysis of concentration standards and generation of a standard curve in the MultiCode-RTx JAK2 V617F assay.

FIGS. 3A and 3B are graphs comparing the ΔCt obtained for three different primer systems to detect wild-type and mutant JAK2 nucleic acids.

### DETAILED DESCRIPTION

Disclosed herein are methods and materials for identifying target nucleic acids and distinguishing among wild-type and mutant nucleic acids. Specifically, the methods disclosed herein can be used to identify JAK2 nucleic acids in a sample.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, “about” means plus or minus 10% unless otherwise indicated.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. For non-natural bases, as described herein, the base-pairing rules include the formation of hydrogen bonds in a manner similar to the Watson-Crick base pairing rules or by hydrophobic, entropic, or van der Waals forces. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

As used herein, a “fragment” means a polynucleotide that is at least about 30, 50, 100, 200, 300, 400, 500, or 1000 nucleotides in length. In one embodiment, the methods may be used to detect a fragment of JAK2 nucleic acid. In other words, the methods may be used to detect a fragment of wt JAK2 nucleic acid and/or a fragment of mutant JAK2 nucleic acid that is at least about 30, 50, 100, 200, 300, 400, 500, or 1000 nucleotides in length.

As used herein, “JAK2 nucleic acid” means nucleic acid that encodes the human JAK2 kinase or a fragment thereof. “JAK2 nucleic acid” may include genomic DNA, mRNA, and/or cDNA. Typically, the primers used in the reaction mixtures described herein are complementary to one or more exons of the wt JAK2 gene or a mutant thereof. For example, genomic JAK2 nucleic acid may be amplified using a first primer that is complementary to an exon sequence of the JAK2 gene and a second primer that is complementary to an intron or exon sequence of the JAK2 gene. “JAK2 nucleic acid” may include “wild-type JAK2 nucleic acid” and “mutant JAK2 nucleic acid.”

“Wild-type JAK2 nucleic acid” or “wt JAK2 nucleic acid” means nucleic acid that encodes the wild-type JAK2 kinase or a fragment thereof, including the human wt JAK2 gene or the expressed mRNA or a cDNA copy of the expressed mRNA. The cDNA sequence of JAK2 mRNA (SEQ ID NO:1) (GenBank accession no. gi:13325062, Locus NM_{—}004972) is shown in Table 1. Table 2 provides the amino acid sequence of the human JAK2 kinase (SEQ ID NO:2). Accordingly, in one embodiment, the wt JAK2 nucleic acid is SEQ ID NO:1 or a fragment thereof. The primers used in the reaction mixture described herein may be complementary to SEQ ID NO:1 (or its complement) or may comprise a contiguous sequence of SEQ ID NO:1 (e.g., at least 10, 15, 20, 25, 30, 35, 40, 45, or 50 contiguous nucleotides).

TABLE 1
Nucleotide Sequence of JAK2 (SEQ ID NO: 1)

CTGCAGGAAGGAGAGAGGAAGAGGAGCAGAAGGGGGCAGCAGCGGACGCC
GCTAACGGCCTCCCTCGGCGCTGACAGGCTGGGCCGGCGCCCGGCTCGCT
TGGGTGTTCGCGTCGCCACTTCGGCTTCTCGGCCGGTCGGGCCCCTCGGC
CCGGGCTTGCGGCGCGCGTCGGGGCTGAGGGCTGCTGCGGCGCAGGGAGA
GGCCTGGTCCTCGCTGCCGAGGGATGTGAGTGGGAGCTGAGCCCACACTG
GAGGGCCCCCGAGGGCCCAGCCTGGAGGTCGTTCAGAGCCGTGCCCGCCC
CGGGGCTTCGCAGACCTTGACCCGCCGGGTAGGAGCCGCCCCTGCGGGCT
CGAGGGCGCGCTCTGGTCGCCCGATCTGTGTAGCCGGTTTCAGAAGCAGG
CAACAGGAACAAGATGTGAACTGTTTCTCTTCTGCAGAAAAAGAGGCTCT
TCCTCCTCCTCCCGCGACGGCAAATGTTCTGAAAAAGACTCTGCATGGGA
ATGGCCTGCCTTACGATGACAGAAATGGAGGGAACATCCACCTCTTCTAT
ATATCAGAATGGTGATATTTCTGGAAATGCCAATTCTATGAAGCAAATAG
ATCCAGTTCTTCAGGTGTATCTTTACCATTCCCTTGGGAAATCTGAGGCA
GATTATCTGACCTTTCCATCTGGGGAGTATGTTGCAGAAGAAATCTGTAT
TGCTGCTTCTAAAGCTTGTGGTATCACACCTGTGTATCATAATATGTTTG
CTTTAATGAGTGAAACAGAAAGGATCTGGTATCCACCCAACCATGTCTTC
CATATAGATGAGTCAACCAGGCATAATGTACTCTACAGAATAAGATTTTA
CTTTCCTCGTTGGTATTGCAGTGGCAGCAACAGAGCCTATCGGCATGGAA
TATCTCGAGGTGCTGAAGCTCCTCTTCTTGATGACTTTGTCATGTCTTAC
CTCTTTGCTCAGTGGCGGCATGATTTTGTGCACGGATGGATAAAAGTACC
TGTGACTCATGAAACACAGGAAGAATGTCTTGGGATGGCAGTGTTAGATA
TGATGAGAATAGCCAAAGAAAACGATCAAACCCCACTGGCCATCTATAAC
TCTATCAGCTACAAGACATTCTTACCAAAATGTATTCGAGCAAAGATCCA
AGACTATCATATTTTGACAAGGAAGCGAATAAGGTACAGATTTCGCAGAT
TTATTCAGCAATTCAGCCAATGCAAAGCCACTGCCAGAAACTTGAAACTT
AAGTATCTTATAAATCTGGAAACTCTGCAGTCTGCCTTCTACACAGAGAA
ATTTGAAGTAAAAGAACCTGGAAGTGGTCCTTCAGGTGAGGAGATTTTTG
CAACCATTATAATAACTGGAAACGGTGGAATTCAGTGGTCAAGAGGGAAA
CATAAAGAAAGTGAGACACTGACAGAACAGGATTTACAGTTATATTGCGA
TTTTCCTAATATTATTGATGTCAGTATTAAGCAAGCAAACCAAGAGGGTT
CAAATGAAAGCCGAGTTGTAACTATCCATAAGCAAGATGGTAAAAATCTG
GAAATTGAACTTAGCTCATTAAGGGAAGCTTTGTCTTTCGTGTCATTAAT
TGATGGATATTATAGATTAACTGCAGATGCACATCATTACCTCTGTAAAG
AAGTAGCACCTCCAGCCGTGCTTGAAAATATACAAAGCAACTGTCATGGC
CCAATTTCGATGGATTTTGCCATTAGTAAACTGAAGAAAGCAGGTAATCA
GACTGGACTGTATGTACTTCGATGCAGTCCTAAGGACTTTAATAAATATT
TTTTGACTTTTGCTGTCGAGCGAGAAAATGTCATTGAATATAAACACTGT
TTGATTACAAAAAATGAGAATGAAGAGTACAACCTCAGTGGGACAAAGAA
GAACTTCAGCAGTCTTAAAGATCTTTTGAATTGTTACCAGATGGAAACTG
TTCGCTCAGACAATATAATTTTCCAGTTTACTAAATGCTGTCCCCCAAAG
CCAAAAGATAAATCAAACCTTCTAGTCTTCAGAACGAATGGTGTTTCTGA
TGTACCAACCTCACCAACATTACAGAGGCCTACTCATATGAACCAAATGG
TGTTTCACAAAATCAGAAATGAAGATTTGATATTTAATGAAAGCCTTGGC
CAAGGCACTTTTACAAAGATTTTTAAAGGCGTACGAAGAGAAGTAGGAGA
CTACGGTCAACTGCATGAAACAGAAGTTCTTTTAAAAGTTCTGGATAAAG
CACACAGAAACTATTCAGAGTCTTTCTTTGAAGCAGCAAGTATGATGAGC
AAGCTTTCTCACAAGCATTTGGTTTTAAATTATGGAGTATGTGTCTGTGG
AGACGAGAATATTCTGGTTCAGGAGTTTGTAAAATTTGGATCACTAGATA
CATATCTGAAAAAGAATAAAAATTGTATAAATATATTATGGAAACTTGAA
GTTGCTAAACAGTTGGCATGGGCCATGCATTTTCTAGAAGAAAACACCCT
TATTCATGGGAATGTATGTGCCAAAAATATTCTGCTTATCAGAGAAGAAG
ACAGGAAGACAGGAAATCCTCCTTTCATCAAACTTAGTGATCCTGGCATT
AGTATTACAGTTTTGCCAAAGGACATTCTTCAGGAGAGAATACCATGGGT
ACCACCTGAATGCATTGAAAATCCTAAAAATTTAAATTTGGCAACAGACA
AATGGACTTTTGGTACCACTTTGTGGGAAATCTGCAGTGGAGGAGATAAA
CCTCTAAGTGCTCTGGATTCTCAAAGAAAGCTACAATTTTATGAAGATAG
GCATCAGCTTCCTGCACCAAAGTGGGCAGAATTAGCAAACCTTATAAATA
ATTGTATGGATTATGAACCAGATTTCAGGCCTTCTTTCAGAGCCATCATA
CGAGATCTTAACAGTTTGTTTACTCCAGATTATGAACTATTAACAGAAAA
TGACATGTTACCAAATATGAGGATAGGTGCCCTAGGGTTTTCTGGTGCCT
TTGAAGACCGGGATCCTACACAGTTTGAAGAGAGACATTTGAAATTTCTA
CAGCAACTTGGCAAGGGTAATTTTGGGAGTGTGGAGATGTGCCGGTATGA
CCCTCTACAGGACAACACTGGGGAGGTGGTCGCTGTAAAAAAGCTTCAGC
ATAGTACTGAAGAGCACCTAAGAGACTTTGAAAGGGAAATTGAAATCCTG
AAATCCCTACAGCATGACAACATTGTAAAGTACAAGGGAGTGTGCTACAG
TGCTGGTCGGCGTAATCTAAAATTAATTATGGAATATTTACCATATGGAA
GTTTACGAGACTATCTTCAAAAACATAAAGAACGGATAGATCACATAAAA
CTTCTGCAGTACACATCTCAGATATGCAAGGGTATGGAGTATCTTGGTAC
AAAAAGGTATATCCACAGGGATCTGGCAACGAGAAATATATTGGTGGAGA
ACGAGAACAGAGTTAAAATTGGAGATTTTGGGTTAACCAAAGTCTTGCCA
CAAGACAAAGAATACTATAAAGTAAAAGAACCTGGTGAAAGTCCCATATT
CTGGTATGCTCCAGAATCACTGACAGAGAGCAAGTTTTCTGTGGCCTCAG
ATGTTTGGAGCTTTGGAGTGGTTCTGTATGAACTTTTCACATACATTGAG
AAGAGTAAAAGTCCACCAGCGGAATTTATGCGTATGATTGGCAATGACAA
ACAAGGACAGATGATCGTGTTCCATTTGATAGAACTTTTGAAGAATAATG
GAAGATTACCAAGACCAGATGGATGCCCAGATGAGATCTATATGATCATG
ACAGAATGCTGGAACAATAATGTAAATCAACGCCCCTCCTTTAGGGATCT
AGCTCTTCGAGTGGATCAAATAAGGGATAACATGGCTGGATGAAAGAAAT
GACCTTCATTCTGAGACCAAAGTAGATTTACAGAACAAAGTTTTATATTT
CACATTGCTGTGGACTATTATTACATATATCATTATTATATAAATCATGA
TGCTAGCCAGCAAAGATGTGAAAATATCTGCTCAAAACTTTCAAAGTTTA
GTAAGTTTTTCTTCATGAGGCCACCAGTAAAAGACATTAATGAGAATTCC
TTAGCAAGGATTTTGTAAGAAGTTTCTTAAACATTGTCTGTTAACATCAC
TCTTGTCTGGCAAAAGAAAAAAAATAGACTTTTTCAACTCAGCTTTTTGA
GACCTGAAAAAATTATTATGTAAATTTTGCAATGTTAAAGATGCACAGAA
TATGTATGTATAGTTTTTACCACAGTGGATGTATAATACCTTGGCATCTT
GTGTGATGTTTTACACACATGAGGGCTGGTGTTCATTAATACTGTTTTCT
AATTTTTCCATAGTTAATCTATAATTAATTACTTCACTATACAAACAAAT
TAAGATGTTCAGATAATTGAATAAGTACCTTTGTGTCCTTGTTCATTTAT
ATCGCTGGCCAGCATTATAAGCAGGTGTATACTTTTAGCTTGTAGTTCCA
TGTACTGTAAATATTTTTCACATAAAGGGAACAAATGTCTAGTTTTATTT
GTATAGGAAATTTCCCTGACCCTAAATAATACATTTTGAAATGAAACAAG
CTTACAAAGATATAATCTATTTTATTATGGTTTCCCTTGTATCTATTTGT
GGTGAATGTGTTTTTTAAATGGAACTATCTCCAAATTTTTCTAAGACTAC
TATGAACAGTTTTCTTTTAAAATTTTGAGATTAAGAATGCCAGGAATATT
GTCATCCTTTGAGCTGCTGACTGCCAATAACATTCTTCGATCTCTGGGAT
TTATGCTCATGAACTAAATTTAAGCTTAAGCCATAAAATAGATTAGATTG
TTTTTTAAAAATGGATAGCTCATTAAGAAGTGCAGCAGGTTAAGAATTTT
TTCCTAAAGACTGTATATTTGAGGGGTTTCAGAATTTTGCATTGCAGTCA
TAGAAGAGATTTATTTCCTTTTTAGAGGGGAAATGAGGTAAATAAGTAAA
AAAGTATGCTTGTTAATTTTATTCAAGAATGCCAGTAGAAAATTCATAAC
GTGTATCTTTAAGAAAAATGAGCATACATCTTAAATCTTTTCAATTA

TABLE 2
Amino Acid Sequence of JAK2 (SEQ ID NO: 2)

MGMACLTMTEMEGTSTSSIYQNGDISGNANSMKQIDPVLQVYLYHSLGKS
EADYLTFPSGEYVAEEICIAASKACGITPVYHNMFALMSETERIWYPPNH
VFHIDESTRHNVLYRIRFYFPRWYCSGSNRAYRHGISRGAEAPLLDDFVM
SYLFAQWRHDFVHGWIKVPVTHETQEECLGMAVLDMMRIAKENDQTPLAI
YNSISYKTFLPKCIRAKIQDYHILTRKRIRYRFRRFIQQFSQCKATARNL
KLKYLINLETLQSAFYTEKFEVKEPGSGPSGEEIFATIIITGNGGIQWSR
GKHKESETLTEQDLQLYCDFPNIIDVSIKQANQEGSNESRVVTIHKQDGK
NLEIELSSLREALSFVSLIDGYYRLTADAHHYLCKEVAPPAVLENIQSNC
HGPISMDFAISKLKKAGNQTGLYVLRCSPKDFNKYFLTFAVERENVIEYK
HCLITKNENEEYNLSGTKKNFSSLKDLLNCYQMETVRSDNIIFQFTKCCP
PKPKDKSNLLVFRTNGVSDVPTSPTLQRPTHMNQMVFHKIRNEDLIFNES
LGQGTFTKIFKGVRREVGDYGQLHETEVLLKVLDKAHRNYSESFFEAADM
MSKLSHKHLVLNYGVCVCGDENILVQEFVKFGSLDTYLKKNKNCINILWK
LEVAKQLAWAMHFLEENTLIHGNVCAKNILLIREEDRKTGNPPFIKLSDP
GISITVLPKDILQERIPWVPPECIENPKNLNLATDKWSFGTTLWEICSGG
DKPLSALDSQRKLQFYEDRHQLPAPKWAELANLINNCMDYEPDFRPSFRA
IIRDLNSLFTPDYELLTENDMLPNMRIGALGFSGAFEDRDPTQFEERHLK
FLQQLGKGNFGSVEMCRYDPLQDNTGEVVAVKKLQHSTEEHLRDFEREIB
ILKSLQHDNIVKYKGVCYSAGRRNLKLIMEYLPYGSLRDYLQKHKERIDH
IKLLQYTSQICKGMEYLGTKRYIHRDLATRNILVENENRVKIGDFGLTKV
LPQDKEYYKVKEPGESPIFWYAPESLTESKFSVASDVWSFGVVLYELFTY
IEKSKSPPAEFMRMIGNKDQGQMIVFHLIELLKNNGRLPRPDGCPDEIYM
IMTECWNNNVNQRPSFRDLALRVDQIRDNMAG

“Mutant JAK2 nucleic acid” means a nucleic acid that encodes a mutant JAK2 kinase or a fragment thereof, wherein polypeptide has one or more mutations relative to wt JAK2 nucleic acid. Typically, mutant JAK2 nucleic acid will have one or more mutations relative to wt JAK2 nucleic within an exon of the JAK2 gene. For example, mutant JAK2 nucleic acid may have one or more mutations relative to the sequence of SEQ ID NO:1. The mutation may effect a change in the amino acid sequence of the encoded polypeptide or the mutation may be silent. Typically, the mutation effects a change in the amino acid sequence of the encoded polypeptide. For example, a mutant JAK2 nucleic acid may encode a polypeptide having one or more amino acid substitutions relative to the sequence of SEQ ID NO:2. Table 3 shows the nucleotide sequence of the mutant JAK2 encoding a V617F mutation (G2343T, underlined). Relative to SEQ ID NO:1, nucleotides 2161 to 2520 are shown. Accordingly, in one embodiment, “mutant JAK2 nucleic acid” may comprise the polynucleotide sequence of SEQ ID NO:3 or a fragment thereof. For example, the mutant JAK2 nucleic acid may include a G to T transversion at nucleotide position 2343 of SEQ ID NO:1.

TABLE 3
Sequence Encoding V617F Mutant JAK2 (SEQ ID NO: 3)

| | |
|---|---|
| 2161 | TTACAAAGATTTTTAAAGGCGTACGAAGAGAAGTAGGAGACTACG |
| | GTCAACTGCATGAAA |
| 2221 | CAGAAGTTCTTTTAAAAGTTCTGGATAAAGCACACAGAAACTATT |
| | CAGAGTCTTTCTTTG |
| 2281 | AAGCAGCAAGTATGATGAGCAAGCTTTCTCACAAGCATTTGGTTT |
| | TAAATTATGGAGTAT |
| 2341 | GTTTCTGTGGAGACGAGAATATTCTGGTTCAGGAGTTTGTAAAAT |
| | TTGGATCACTAGATA |
| 2401 | CATATCTGAAAAAGAATAAAAATTGTATAAATATATTATGGAAAC |
| | TTGAAGTTGCTAAAC |
| 2461 | AGTTGGCATGGGCCATGCATTTTCTAGAAGAAAACACCCTTATTC |
| | ATGGGAATGTATGTG |

In further embodiments, the mutant JAK2 nucleic acid may encode a polypeptide having one or more of the following DNA alterations relative to SEQ ID NO:1: (a) 1627-1632del6 (predicted E543-D544del amino acid change); (b) 1606-1638dup33 (predicted V536-I546dup11 amino acid change); (c) 1624-1629del6 (predicted N542-E543del amino acid change); (d) 1608-1640dup133 (predicted F537-I546dup10+F547L amino acid change); (e) 1622-1627del6 (predicted R541-N542-E543delinsK amino acid change); (f) 1620-1621del2, 1626-1629del4 (predicted I540-E543delinskMK amino acid change); (g) 1611-1616del6 (predicted F537-K539delinsL amino acid change); (h) 1611-1616del6 (predicted F537-K539delinsL amino acid change); and (i) 1613-1615del3, A1616T (predicted H538-K439delinsL amino acid change).

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. RNA may be used in the methods described herein and/or may be converted to cDNA by reverse-transcription for use in the methods described herein.

As used herein, the term “sample” is used in its broadest sense. A sample may include a bodily tissue or a bodily fluid including but not limited to blood (or a fraction of blood such as plasma or serum), lymph, mucus, tears, urine, and saliva. A sample may include an extract from a cell, a chromosome, organelle, or a virus. A sample may be a “cell-free” sample, meaning that the volume of cells in the sample are less than about 2% of the total sample volume (preferably less than about 1% of the total sample volume). A sample may comprise DNA (e.g., genomic DNA), RNA (e.g., mRNA), and cDNA, any of which may be amplified to provide amplified nucleic acid. For example, a sample may include nucleic acid in solution or bound to a substrate (e.g., as part of a microarray). A sample may comprise material obtained from an environmental locus (e.g., a body of water, soil, and the like) or material obtained from a fomite (i.e., an inanimate object that serves to transfer pathogens from one host to another). A sample may be obtained from any patient. In particular, a sample may be obtained from a patient having or suspected to be at risk for developing a myeloproliferative disorder such as PV, ET, or IMF.

As used herein, “target nucleic acid” refers to a nucleic acid containing a nucleic acid sequence, suspected to be in a sample and to be detected or quantified in a method or system as disclosed herein. Target nucleic acids contain the target nucleic acid sequences that are actually assayed during an assay procedure. The target can be directly or indirectly assayed. In at least some embodiments, the target nucleic acid, if present in the sample, is used as a template for amplification according to the methods disclosed herein. Target nucleic acid may include JAK2 nucleic acid including wt JAK2 nucleic acid and mutant JAK2 nucleic acid.

### Oligonucleotides and Specific Primers

An oligonucleotide is a nucleic acid that includes at least two nucleotides. Oligonucleotides used in the methods disclosed herein typically include at least about ten (10) nucleotides and more typically at least about fifteen (15) nucleotides. Oligonucleotides for the methods disclosed herein may include about 10-25 nucleotides.

Oligonucleotides as described herein typically are capable of forming hydrogen bonds with oligonucleotides having a complementary base sequence. These bases may include the natural bases such as A, G, C, T and U, as well as artificial bases such as deaza-G. As described herein, a first sequence of an oligonucleotide is described as being 100% complementary with a second sequence of an oligonucleotide when the consecutive bases of the first sequence (read 5′ to 3′) follow the Watson-Crick rule of base pairing as compared to the consecutive bases of the second sequence (read 3′ to 5′). An oligonucleotide may include nucleotide substitutions. For example, an artificial base may be used in place of a natural base such that the artificial base exhibits a specific interaction that is similar to the natural base.

An oligonucleotide may be designed to function as a primer. As used herein, a “primer” for amplification is an oligonucleotide that is complementary to a target nucleotide sequence and leads to addition of nucleotides to the 3′ end of the primer in the presence of a DNA or RNA polymerase. The 5 nucleotides at the 3′ terminus of a primer should generally be identical to the target sequence at a corresponding nucleotide position for optimal expression and/or amplification. The term “primer” includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA encoding a polypeptide. A “reverse primer” is complementary to the sense-strand of dsDNA encoding a polypeptide. Primers which are suitable for amplifying a target nucleic acid are generally capable of specifically hybridizing to the target nucleic acid.

A primer that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST).

A primer that is specific for a target nucleic acid will “hybridize” to the target nucleic acid under suitable conditions. As used herein, “hybridization” or “hybridizing” refers to the process by which a oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art. Stringent hybridization conditions will be those in which the salt concentration is less than about 1.5 M Na ion, typically about 0.01 to 1.0 M Na ion concentration (or other salts) at pH 7.0 to 8.3 and the temperature is at least about 30° C. for short oligonucleotides (e.g., 10 to 50 nucleotides) and at least about 60° C. for long oligonucleotides (e.g., greater than 50 nucleotides). Stringent conditions may also be achieved with the addition of destabilizing agents such as formamide.

In some embodiments of the methods disclosed herein, a sample is suspected to contain a mutant JAK2 nucleic acid. The mutant JAK2 nucleic acid typically differs from wt JAK2 nucleic acids of by at least a single nucleotide base. A first specific primer for the mutant JAK2 nucleic acid and a second specific primer wt JAK2 nucleic acid are added to the sample, along with a universal primer and a non-natural nucleotide base having a label. The sequence of each specific primer may differ from another at one terminus or near a terminus (e.g., within 1 base, 2 bases, 3 bases, or 4 bases from the terminus). The sequence of each specific primer may differ from another (e.g., by a single nucleotide, by two nucleotides, or by three nucleotides). Each specific primer may include an identical non-natural nucleotide base and a label (e.g., a fluorescent label, a radiolabel, and an enzyme label). Each label may be different from the other. For example, one label may be fluorescein (FAM) and the other label may be hexachlorofluorescein (HEX).

In some embodiments, the specific primers comprise a 5′ tail sequence. Typically, the 5′ tail sequence comprises nucleotides non-complementary to the target sequence. The tails may be designed to improve the specificity of the primers by reducing mispriming during PCR, i.e., the tail sequences can be designed to add about 10° C. to the Tₘ of the specific primers. For example, the annealing temperature used in the first 1 to 5 cycles of PCR with tailed primers may be about 5° C. to 15° C. lower than the annealing temperature in subsequent PCR cycles. In one embodiment, the 5′ tail sequence comprises about 1-5, about 1-10, about 2-10, about 3-10, about 4-10, about 5-10 or more nucleotides, which are not capable of specifically hybridizing to the target nucleic acid. The tail sequence may comprise one or more non-standard bases. In a suitable embodiment, the 5′ tail sequences of the first primer and the second primer are different so as to maintain an annealing temperature differential between the two primers. The annealing temperature differential between the 5′ tails of the first primer and the second primer may be from about 1° C. to 10° C., from about 1° C. to about 7° C., or from about 1° C. to about 5° C.

As will be apparent from the discussion herein, the relative sizes of the specific primers, as well as the amplified portion of the target nucleic acids, will vary depending upon the particular application. Further, the relative location of the primers along the target nucleic acid will vary. Additionally, the location of the non-natural base and labels used in the methods disclosed herein will vary depending upon application.

Additionally, the length of the primer can affect the temperature at which the primer will hybridize to the target nucleic acid. Generally, a longer primer will form a sufficiently stable hybrid to the target nucleic acid sequence at a higher temperature than will a shorter primer. Further, the presence of high proportion of G or C or of particular non-natural bases in the primer can enhance the stability of a hybrid formed between the primer and the target nucleic acid. This increased stability can be due to, for example, the presence of three hydrogen bonds in a G-C interaction or other non-natural base pair interaction compared to two hydrogen bonds in an A-T interaction.

Stability of a nucleic acid duplex can be estimated or represented by the melting temperature, or “Tₘ.” The Tₘ of a particular nucleic acid duplex under specified conditions is the temperature at which 50% of the population of the nucleic acid duplexes dissociate into single-stranded nucleic acid molecules. The Tₘ of a particular nucleic acid duplex can be predicted by any suitable method. Suitable methods for determining the Tₘ of a particular nucleic acid duplex include, for example, software programs. Primers suitable for use in the methods and kits disclosed herein can be predetermined based on the predicted Tₘ of an oligonucleotide duplex that comprises the primer.

When the first primer and second primer are annealed to the target nucleic acid, a gap exists between the 3′ terminal nucleotide of the first primer and the 3′ terminal nucleotide of the second primer. The gap comprises a number of nucleotides of the target nucleic acid. The gap can be any number of nucleotides provided that the polymerase can effectively incorporate nucleotides into an elongating strand to fill the gap during a round of the PCR reaction (e.g., a round of annealing, extension, denaturation). Typically, a polymerase can place about 30 to about 100 bases per second. Thus, the maximum length of the gap between primers depends upon the amount of time within a round of PCR where the temperature is in a range in which the polymerase is active and the primers are annealed.

The oligonucleotides may include at least one non-natural nucleotide. For example, the oligonucleotides may include at least one nucleotide that includes a nucleobase other than A, C, G, T, or U (e.g., iC or iG). Where the oligonucleotide is used as a primer for PCR, the amplification mixture may include at least one nucleotide that is labeled with a quencher (e.g., Dabcyl). The labeled nucleotide may include at least one non-natural nucleotide. For example, the labeled nucleotide may include at least one nucleobase that is not A, C, G, T, or U (e.g., iC or iG).

In some embodiments, the oligonucleotide may be designed to avoid forming an intramolecular structure such as a hairpin. In other embodiments, the oligonucleotide may be designed to form an intramolecular structure such as a hairpin. For example, the oligonucleotide may be designed to form a hairpin structure that is altered after the oligonucleotide hybridizes to a target nucleic acid, and optionally, after the target nucleic acid is amplified using the oligonucleotide as a primer (See, e.g., U.S. Pat. No. 5,928,869).

The oligonucleotide may be labeled with a fluorophore that exhibits quenching when incorporated in an amplified product as a primer. In other embodiments, the oligonucleotide may emit a detectable signal after the oligonucleotide is incorporated in an amplified product as a primer. Such primers are known in the art (e.g., LightCycler primers, Amplifluor® Primers, Scorpion® Primers and Lux™ Primers). The fluorophore used to label the oligonucleotide may emit a signal when intercalated in double-stranded nucleic acid. As such, the fluorophore may emit a signal after the oligonucleotide is used as a primer for amplifying the nucleic acid.

The disclosed methods may be performed with any suitable number of oligonucleotides. Where a plurality of oligonucleotides are used (e.g., two or more oligonucleotides), different oligonucleotides may be labeled with different fluorescent dyes capable of producing a detectable signal. In some embodiments, oligonucleotides are labeled with at least one of two different fluorescent dyes. In further embodiments, oligonucleotides are labeled with at least one of three different fluorescent dyes. In some embodiments, each different fluorescent dye emits a signal that can be distinguished from a signal emitted by any other of the different fluorescent dyes that are used to label the oligonucleotides. For example, the different fluorescent dyes may have wavelength emission maximums all of which differ from each other by at least about 5 nm (preferably by least about 10 nm). In some embodiments, each different fluorescent dye is excited by different wavelength energies. For example, the different fluorescent dyes may have wavelength absorption maximums all of which differ from each other by at least about 5 nm (preferably by at least about 10 nm).

In some embodiments, the primers used in the reactions described herein may be complementary to SEQ ID NO: 1 or SEQ ID NO:3 (or their complements) or may comprise a contiguous sequence of SEQ ID NO: 1 or SEQ ID NO:3 (e.g., at least 10, 15, 20, 25, 30, 35, 40, 45, or 50 contiguous nucleotides). Exemplary primers for the wild-type and mutant JAK2 nucleic acids are shown in Tables 4 and 5. The primers may comprise a non-natural base, such as iso-C (X) or iso-G (Y). In some embodiments, the allele specific primers, i.e., the primers which discriminate between JAK2 mutant and wild-type, are reverse primers based on the orientation of the JAK2 coding sequence (Table 4). In some embodiments, the allele specific primers are forward primers (Table 5).

TABLE 4
Reverse ASP System Primer Sequences

| Primer | | | |
|---|---|---|---|
| Name | Description | Sequence (5′ to 3′) | SEQ ID NO: |
| DM1168 | Common | ATGATGAGCAAGCTTTCTCACAAGC | SEQ ID NO: 8 |
| | Fwd Primer | | |
| DM1169 | Common | AGCAAGTATGATGAGCAAGCTTTCTC | SEQ ID NO: 9 |
| | Fwd Primer | | |
| DM1170 | Common | GCAGCAAGTATGATGAGCAAGCTTT | SEQ ID NO: 10 |
| | Fwd Primer | | |
| DM1171 | Common | GCTTTCTCACAAGCATTTGGTTT | SEQ ID NO: 11 |
| | Fwd Primer | | |
| DM1172 | Common | CACAAGCATTTGGTTTTAAATTATGGAGTAT | SEQ ID NO: 12 |
| | Fwd Primer | | |
| DM1173 | Common | ATGATGAGCAAGCTTTCTCACA | SEQ ID NO: 13 |
| | Fwd Primer | | |
| DM1174 | Common | TCTCACAAGCATTTGGTTTTAAATTATGGAGT | SEQ ID NO: 14 |
| | Fwd Primer | | |
| DM1160 | Rev WT | XTGTCCACTCGTCTCCACAGACA | SEQ ID NO: 15 |
| | Primer | YTGTCCACTCGTCTCCACAGACA | SEQ ID NO: 16 |
| | | CTCGTCTCCACAGACA | SEQ ID NO: 17 |
| DM1161 | Rev WT | XTGTCCACTCGTCTCCACAGAC | SEQ ID NO: 18 |
| | Primer | YTGTCCACTCGTCTCCACAGAC | SEQ ID NO: 19 |
| | | CTCGTCTCCACAGAC | SEQ ID NO: 20 |
| DM1162 | Rev WT | XCACTCTCGTCTCCACAGGCA | SEQ ID NO: 21 |
| | Primer | YCACTCTCGTCTCCACAGGCA | SEQ ID NO: 22 |
| | | CTCTCGTCTCCACAGGCA | SEQ ID NO: 23 |
| DM1163 | Rev WT | XCACTCTCGTCTCCACGGACA | SEQ ID NO: 24 |
| | Primer | YCACTCTCGTCTCCACGGACA | SEQ ID NO: 25 |
| | | CTCTCGTCTCCACGGACA | SEQ ID NO: 26 |
| DM1164 | Rev Mut | XACAGGTCTCGTCTCCACAGAAA | SEQ ID NO: 27 |
| | Primer | YACAGGTCTCGTCTCCACAGAAA | SEQ ID NO: 28 |
| | | CTCGTCTCCACAGAAA | SEQ ID NO: 29 |
| DM1165 | Rev Mut | XACAGGTCTCGTCTCCACAGAA | SEQ ID NO: 30 |
| | Primer | YACAGGTCTCGTCTCCACAGAA | SEQ ID NO: 31 |
| | | CTCGTCTCCACAGAA | SEQ ID NO: 32 |
| DM1166 | Rev Mut | XGGTCTCTCGTCTCCACAGGAA | SEQ ID NO: 33 |
| | Primer | YGGTCTCTCGTCTCCACAGGAA | SEQ ID NO: 34 |
| | | CTCTCGTCTCCACAGGAA | SEQ ID NO: 35 |
| DM1167 | Rev Mut | XGGTACTCTCGTCTCCACGGAAA | SEQ ID NO: 36 |
| | Primer | YGGTACTCTCGTCTCCACGGAAA | SEQ ID NO: 37 |
| | | ACTCTCGTCTCCACGGAAA | SEQ ID NO: 38 |

TABLE 5
Forward ASP System Primer Sequences

| Primer | | | |
|---|---|---|---|
| Name | Description | Sequence (5′ to 3′) | SEQ ID NO: |
| DM1175 | Fwd WT | XACAGGTTTTTAAATTATGGAGTATGTGT | SEQ ID NO: 39 |
| | primer | YACAGGTTTTTAAATTATGGAGTATGTGT | SEQ ID NO: 40 |
| | | TTTTAAATTATGGAGTATGTGT | SEQ ID NO: 41 |
| DM1176 | Fwd WT | XACAGGTGTTTTAAATTATGGAGTATGTG | SEQ ID NO: 42 |
| | Primer | YACAGGTGTTTTAAATTATGGAGTATGTG | SEQ ID NO: 43 |
| | | GTTTTAAATTATGGAGTATGTG | SEQ ID NO: 44 |
| DM1177 | Fwd Mut | XTGTCCAGTTTTAAATTATGGAGTATGTTT | SEQ ID NO: 45 |
| | Primer | YTGTCCAGTTTTAAATTATGGAGTATGTTT | SEQ ID NO: 46 |
| | | GTTTTAAATTATGGAGTATGTTT | SEQ ID NO: 47 |
| DM1178 | Fwd Mut | XTGTCCAGTTTTAAATTATGGAGTATGTT | SEQ ID NO: 48 |
| | Primer | YTGTCCAGTTTTAAATTATGGAGTATGTT | SEQ ID NO: 49 |
| | | GTTTTAAATTATGGAGTATGTT | SEQ ID NO: 50 |
| DM1179 | Common Rev | GCCTGTAGTTTTACTTACTCTCGTCT | SEQ ID NO: 51 |
| | Primer | | |
| DM1180 | Common Rev | AGCCTGTAGTTTTACTTACTCTCG | SEQ ID NO: 52 |
| | Primer | | |
| DM1181 | Common Rev | AGCATTAGAAAGCCTGTAGT | SEQ ID NO: 53 |
| | Primer | | |
| DM1182 | Common Rev | GTAGTTTTACTTACTCTCGTCTCCAC | SEQ ID NO: 54 |
| | Primer | | |
| DM1183 | Common Rev | TGTAGTTTTACTTACTCTCGTCTCCACAGA | SEQ ID NO: 55 |
| | Primer | | |
| BAK328 | Fwd WT | XCCAGGAGGTTTTAAATTATGGAGTATGTG | SEQ ID NO: 5 |
| | Primer | YCCAGGAGGTTTTAAATTATGGAGTATGTG | SEQ ID NO: 56 |
| | | GGTTTTAAATTATGGAGTATGTG | SEQ ID NO: 57 |
| BAK329 | Fwd Mut | XGGTCCTGGTTTTAAATTATGGAGTATGTT | SEQ ID NO: 4 |
| | Primer | YGGTCCTGGTTTTAAATTATGGAGTATGTT | SEQ ID NO: 58 |
| | | TGGTTTTAAATTATGGAGTATGTT | SEQ ID NO: 59 |
| BAK327 | Common Rev | GAACCAGAATATTCTCGTCTCCACAG | SEQ ID NO: 6 |
| | Primer | | |
| | Common Rev | CTGTGGAGACGAGAATATTCTGGTT | SEQ ID NO: 7 |
| | Primer | | |

As used herein, “universal primer” refers to a primer that can specifically hybridize to two or more different target nucleic acids in a sample (e.g., 3 or more, 4 or more, 5 or more, 10 or more, 15 or more, or 25 or more different target nucleic acids in a sample). A “universal primer” may hybridize to a region of the different target nucleic acids that is identical or that has substantial identity to provide for specific hybridization of the “universal primer” to the different target nucleic acids. A “universal primer” may be complementary to a nucleic acid sequence that is common to all the JAK2 nucleic acids (wild-type and mutant) in a sample.

### Amplification

Disclosed herein are methods for detecting a target nucleic acid that may utilize PCR. The methods may involve a polymerase, a first primer, a second primer, and optionally, a third primer. Traditional PCR methods include the following steps: denaturation, or melting of double-stranded nucleic acids; annealing of primers; and extension of the primers using a polymerase. This cycle is repeated by denaturing the extended primers and starting again. The number of copies of the target sequence in principle grows exponentially. In practice, it typically doubles with each cycle until reaching a plateau at which more primer-template accumulates than the enzyme can extend during the cycle; then the increase in target nucleic acid becomes linear.

In some embodiments, the specific primers are allowed to anneal to the nucleic acids from the wt and/or mutant JAK2 nucleic acids. PCR using a nucleic acid polymerase (as herein described) is performed with chain extension of the annealed specific primer to form a double stranded product. One of the two strands of the product may incorporate a non-natural nucleotide base and the fluorescent label from the specific primer. As PCR progresses, the labeled strand is annealed with the universal primer, which in turn is extended in the opposite direction until the polymerase reaches the non-natural nucleotide base (e.g., isocytosine) and terminates extension with the addition of the complementary non-natural base (e.g., isoguanosine) bearing a fluorescent quencher such as dabcyl. PCR is run for the desired number of cycles to obtain this double-stranded amplification product. As more of the double stranded amplification product accumulates having both a fluorophore and a fluorescent quencher, the fluorescent signal from the specific primer(s) being incorporated into the amplified product will decrease. If only the wild-type JAK2 nucleic acid is present in the sample, only the fluorescent signal associated with the wild-type nucleic acid will decrease. If both wild-type and mutant JAK2 nucleic acids are present, both signals will decrease as the PCR reaction progresses. The relative amounts of wild-type to mutant JAK2 nucleic acids could be determined by comparing the decrease in signals.

The amplification methods described herein may include “real-time monitoring” or “continuous monitoring.” These terms refer to monitoring multiple times during a cycle of PCR, preferably during temperature transitions, and more preferably obtaining at least one data point in each temperature transition. The term “homogeneous detection assay” is used to describe an assay that includes coupled amplification and detection, which may include “real-time monitoring” or “continuous monitoring.” By contrast, “end-point monitoring” refers to the detection of amplification at the termination of a reaction. For example, end-point monitoring may include melting curve analysis and gel electrophoresis and visualization with dyes or autoradiography.

Fast-shot amplification is a modified polymerase chain reaction wherein the extension step, as well as the annealing and melting steps, are very short or eliminated. As used herein, when referring to “steps” of PCR, a step is a period of time during which the reaction is maintained at a desired temperature without substantial fluctuation of that temperature. The time for annealing and melting steps for a typical PCR can range from 30 seconds to 60 seconds. The time for annealing and melting steps for a Fast-shot™ amplification generally can range from about 0 seconds to about 60 seconds. For Fast-shot™ amplification, the annealing and melting steps are typically no more than about 2 seconds, preferably about 1 second or less. When the extension step is eliminated, the temperature is cycled between the annealing and melting steps without including an intermediate extension step between the annealing and melting temperatures.

Additionally, the limit of how quickly the temperature can be changed from the annealing temperature to the melting temperature depends upon the efficiency of the polymerase in incorporating bases onto an extending primer and the number of bases it must incorporate, which is determined by the gap between the primers and the length of the primers.

The number of Fast-shot™ amplification cycles required to determine the presence of a nucleic acid sequence in a sample can vary depending on the number of target molecules in the sample. In one of the examples described below, a total of 37 cycles was adequate to detect as little as 100 target nucleic acid molecules.

PCR may be used to generate an amplification product (i.e., an amplicon) comprising a double-stranded region and a single-stranded region. The double-stranded region may result from extension of the first and second primers. The single-stranded region may result from incorporation of a non-natural base in the second primer of the disclosed methods. A region of the first and/or second primer may not be complementary to the target nucleic acid. Because the non-natural base follows base-pairing rules of Watson and Crick and forms bonds with other non-natural bases, the presence of a non-natural base may maintain a region as a single-stranded region in the amplification product. In an alternative embodiment, the single-stranded region comprises more than one non-natural base. The number of non-natural bases included in the first and/or second primer can be selected as desired.

### Polymerases

Disclosed herein are methods that may utilize an amplification reaction, e.g., the polymerase chain reaction, to detect nucleic acids of interest in a sample (i.e., nucleic acids of the target and non-target species or subspecies). Suitable nucleic acid polymerases include, for example, polymerases capable of extending an oligonucleotide by incorporating nucleic acids complementary to a template oligonucleotide. For example, the polymerase can be a DNA polymerase.

Enzymes having polymerase activity catalyze the formation of a bond between the 3′ hydroxyl group at the growing end of a nucleic acid primer and the 5′ phosphate group of a nucleotide triphosphate. These nucleotide triphosphates are usually selected from deoxyadenosine triphosphate (A), deoxythymidine triphosphate (T), deoxycytosine triphosphate (C) and deoxyguanosine triphosphate (G). However, in at least some embodiments, polymerases useful for the methods disclosed herein also may incorporate non-natural bases using nucleotide triphosphates of those non-natural bases.

Because the relatively high temperatures necessary for strand denaturation during methods such as PCR can result in the irreversible inactivation of many nucleic acid polymerases, nucleic acid polymerase enzymes useful for performing the methods disclosed herein preferably retain sufficient polymerase activity to complete the reaction when subjected to the temperature extremes of methods such as PCR. Preferably, the nucleic acid polymerase enzymes useful for the methods disclosed herein are thermostable nucleic acid polymerases. Suitable thermostable nucleic acid polymerases include, but are not limited to, enzymes derived from thermophilic organisms. Examples of thermophilic organisms from which suitable thermostable nucleic acid polymerase can be derived include, but are not limited to, Thermus aquaticus, Thermus thermophilus, Thermus flavus, Thermotoga neapolitana and species of the Bacillus, Thermococcus, Sulfobus, and Pyrococcus genera. Nucleic acid polymerases can be purified directly from these thermophilic organisms. However, substantial increases in the yield of nucleic acid polymerase can be obtained by first cloning the gene encoding the enzyme in a multicopy expression vector by recombinant DNA technology methods, inserting the vector into a host cell strain capable of expressing the enzyme, culturing the vector-containing host cells, then extracting the nucleic acid polymerase from a host cell strain which has expressed the enzyme. Suitable thermostable nucleic acid polymerases, such as those described above, are commercially available.

Polymerases can “misincorporate” bases during PCR. In other words, the polymerase can incorporate a nucleotide (for example adenine) at the 3′ position on the synthesized strand that does not form canonical hydrogen base pairing with the paired nucleotide (for example, cytosine) on the template nucleic acid strand. The PCR conditions can be altered to decrease the occurrence of misincorporation of bases. For example, reaction conditions such as temperature, salt concentration, pH, detergent concentration, type of metal, concentration of metal, and the like can be altered to decrease the likelihood that polymerase will incorporate a base that is not complementary to the template strand.

As an alternative to using a single polymerase, any of the methods described herein can be performed using multiple enzymes. For example, it will be recognized that RNA can be used as a sample and that a reverse transcriptase can be used to transcribe the RNA to cDNA. The transcription can occur prior to or during PCR amplification.

### Non-Natural Bases

As contemplated in the methods and kits disclosed herein, at least one primer typically comprises at least one non-natural base. DNA and RNA are oligonucleotides that include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytosine (C), and uridine (U). These five bases are “natural bases”. According to the rules of base pairing elaborated by Watson and Crick, the natural bases can hybridize to form purine-pyrimidine base pairs, where G pairs with C and A pairs with T or U. These pairing rules facilitate specific hybridization of an oligonucleotide with a complementary oligonucleotide.

The formation of these base pairs by the natural bases is facilitated by the generation of two or three hydrogen bonds between the two bases of each base pair. Each of the bases includes two or three hydrogen bond donor(s) and hydrogen bond acceptor(s). The hydrogen bonds of the base pair are each formed by the interaction of at least one hydrogen bond donor on one base with a hydrogen bond acceptor on the other base. Hydrogen bond donors include, for example, heteroatoms (e.g., oxygen or nitrogen) that have at least one attached hydrogen. Hydrogen bond acceptors include, for example, heteroatoms (e.g., oxygen or nitrogen) that have a lone pair of electrons.

The natural bases, A, G, C, T, and U, can be derivatized by substitution at non-hydrogen bonding sites to form modified natural bases. For example, a natural base can be derivatized for attachment to a support by coupling a reactive functional group (for example, thiol, hydrazine, alcohol, amine, and the like) to a non-hydrogen bonding atom of the base. Other possible substituents include, for example, biotin, digoxigenin, fluorescent groups, alkyl groups (e.g., methyl or ethyl), and the like.

Non-natural bases, which form hydrogen-bonding base pairs, can also be constructed as described, for example, in U.S. Pat. Nos. 5,432,272; 5,965,364; 6,001,983; 6,037,120; U.S. published application no. 2002/0150900; and U.S. patent application Ser. No. 08/775,401, all of which are incorporated herein by reference. Suitable bases and their corresponding base pairs may include the following bases in base pair combinations (iso-C/iso-G, K/X, H/J, and M/N):

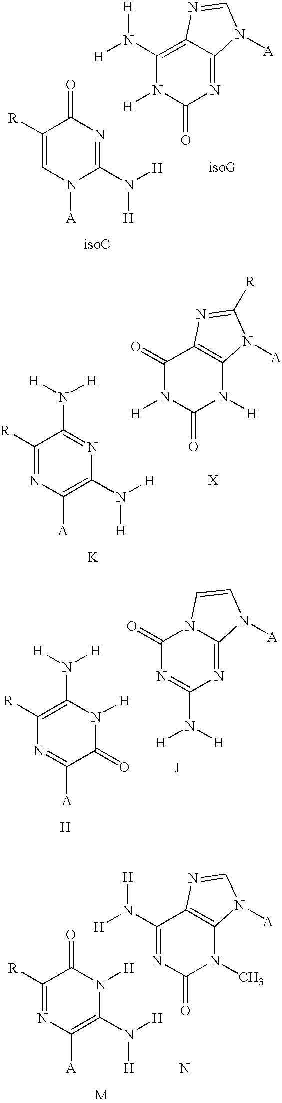

where A is the point of attachment to the sugar or other portion of the polymeric backbone and R is H or a substituted or unsubstituted alkyl group. It will be recognized that other non-natural bases utilizing hydrogen bonding can be prepared, as well as modifications of the above-identified non-natural bases by incorporation of functional groups at the non-hydrogen bonding atoms of the bases.

The hydrogen bonding of these non-natural base pairs is similar to those of the natural bases where two or three hydrogen bonds are formed between hydrogen bond acceptors and hydrogen bond donors of the pairing non-natural bases. One of the differences between the natural bases and these non-natural bases is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-natural bases for use in oligonucleotides include, for example, naphthalene, phenanthrene, and pyrene derivatives as discussed, for example, in Ren et al., J. Am. Chem. Soc. 118, 1671 (1996) and McMinn et al., J. Am. Chem. Soc. 121, 11585 (1999), both of which are incorporated herein by reference. These bases do not utilize hydrogen bonding for stabilization, but instead rely on hydrophobic or van der Waals interactions to form base pairs.

The use of non-natural bases according to the methods disclosed herein is extendable beyond the detection and quantification of nucleic acid sequences present in a sample. For example, non-natural bases can be recognized by many enzymes that catalyze reactions associated with nucleic acids. While a polymerase requires a complementary nucleotide to continue polymerizing an extending oligonucleotide chain, other enzymes do not require a complementary nucleotide. If a non-natural base is present in the template and its complementary non-natural base is not present in the reaction mix, a polymerase will typically stall (or, in some instances, misincorporate a base when given a sufficient amount of time) when attempting to extend an elongating primer past the non-natural base. However, other enzymes that catalyze reactions associated with nucleic acids, such as ligases, kinases, nucleases, polymerases, topoisomerases, helicases, and the like can catalyze reactions involving non-natural bases. Such features of non-natural bases can be taken advantage of, and are within the scope of the presently disclosed methods and kits.

For example, non-natural bases can be used to generate duplexed nucleic acid sequences having a single strand overhang. This can be accomplished by performing a PCR reaction to detect a target nucleic acid in a sample, the target nucleic acid having a first portion and a second portion, where the reaction system includes all four naturally occurring dNTP's, a first primer that is complementary to the first portion of the target nucleic acid, a second primer having a first region and a second region, the first region being complementary to the first portion of the target nucleic acid, and the second region being noncomplementary to the target nucleic acid. The second region of the second primer comprises a non-natural base. The first primer and the first region of the second primer hybridize to the target nucleic acid, if present. Several rounds of PCR will produce an amplification product containing (i) a double-stranded region and (ii) a single-stranded region. The double-stranded region is formed through extension of the first and second primers during PCR. The single-stranded region includes the one or more non-natural bases. The single-stranded region of the amplification product results because the polymerase is not able to form an extension product by polymerization beyond the non-natural base in the absence of the nucleotide triphosphate of the complementary non-natural base. In this way, the non-natural base functions to maintain a single-stranded region of the amplification product.

As mentioned above, the polymerase can, in some instances, misincorporate a base opposite a non-natural base. In this embodiment, the misincorporation takes place because the reaction mix does not include a complementary non-natural base. Therefore, if given sufficient amount of time, the polymerase can, in some cases, misincorporate a base that is present in the reaction mixture opposite the non-natural base.

### Labels

In accordance with the methods and kits disclosed herein, the primers and/or the added non-natural nucleotide base may comprises a label. Nucleotides and oligonucleotides can be labeled by incorporating moieties detectable by spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

As used herein, “labels” are chemical or biochemical moieties useful for labeling a nucleic acid (including a single nucleotide), amino acid, or antibody. “Labels” include fluorescent agents, chemiluminescent agents, chromogenic agents, quenching agents, radionuclides, enzymes, substrates, cofactors, inhibitors, magnetic particles, and other moieties known in the art. “Labels” or “reporter molecules” are capable of generating a measurable signal and may be covalently or noncovalently joined to an oligonucleotide or nucleotide (e.g., a non-natural nucleotide).

A variety of labels which are appropriate for use in the methods and kits, as well as methods for their inclusion in the probe, are disclosed herein and are known in the art. These include, but are not limited to, enzyme substrates, fluorescent dyes, chromophores, chemiluminescent labels, electrochemiluminescent labels, such as ORI-TAG™ (Igen), ligands having specific binding partners, or any other labels that can interact with each other to enhance, alter, or diminish a signal. It is understood that, should the PCR be practiced using a thermocycler instrument, a label should be selected to survive the temperature cycling required in this automated process.

In some embodiments, the primers used in the methods are labeled. For example, the oligonucleotides may include a label that emits a detectable signal. By way of example, the label system may be used to produce a detectable signal based on a change in fluorescence, fluorescence resonance energy transfer (FRET), fluorescence quenching, phosphorescence, bioluminescence resonance energy transfer (BRET), or chemiluminescence resonance energy transfer (CRET).

In some embodiments, two interactive labels may be used on a single oligonucleotide with due consideration given for maintaining an appropriate spacing of the labels on the oligonucleotide to permit the separation of the labels during oligonucleotide hydrolysis. In other embodiments, two interactive labels on different oligonucleotides may be used, such as, for example, the reporter and the second region of the second primer. In this embodiment, the reporter and the second region are designed to hybridize to each other. Again, consideration is given to maintaining an appropriate spacing of the labels between the oligonucleotides when hybridized.

The oligonucleotides and nucleotides (e.g., non-natural nucleotides) of the disclosed methods may be labeled with a “fluorescent dye” or a “fluorophore.” As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following tradenames: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP-Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBF™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF 1; Sodium Green; SpectrumAqua; SpectrumGreen; Spectrum Orange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamineIsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The oligonucleotides and nucleotides of the disclosed methods (e.g., non-natural nucleotides) may be labeled with a quencher. Quenching may include dynamic quenching (e.g., by FRET), static quenching, or both. Suitable quenchers may include Dabcyl. Suitable quenchers may also include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL®” (Anaspec, San Jose, Calif.). Dark quenchers also may include DNP-type non-fluorophores that include a 2,4-dinitrophenyl group.

The oligonucleotides or nucleotides (e.g., non-natural nucleotides) of the present methods may be labeled with a donor fluorophore and an acceptor fluorophore (or quencher dye) that are present in the oligonucleotides at positions that are suitable to permit FRET (or quenching). Labeled oligonucleotides that are suitable for the present methods may include but are not limited to oligonucleotides designed to function as LightCycler primers or probes, Taqman® Probes, Molecular Beacon Probes, Amplifluor® Primers, Scorpion® Primers, and LUX™ Primers.

The labels can be attached to the nucleotides, including non-natural bases, or oligonucleotides directly or indirectly by a variety of techniques. Depending upon the precise type of label used, the label can be located at the 5′ or 3′ end of the reporter, located internally in the reporter's nucleotide sequence, or attached to spacer arms extending from the reporter and having various sizes and compositions to facilitate signal interactions. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite, and can label them using protocols described in, for example, PCR Protocols: A Guide to Methods and Applications, ed. by Innis et al., Academic Press, Inc., 1990, incorporated herein by reference.

Methods for incorporating oligonucleotide functionalizing reagents having one or more sulfhydryl, amino or hydroxyl moieties into the oligonucleotide reporter sequence, typically at the 5′ terminus, are described in U.S. Pat. No. 4,914,210, incorporated herein by reference. For example, 5′ phosphate group can be incorporated as a radioisotope by using polynucleotide kinase and [γ ³²P]ATP to provide a reporter group. Biotin can be added to the 5′ end by reacting an aminothymidine residue, introduced during synthesis, with an N-hydroxysuccinimide ester of biotin.

Labels at the 3′ terminus, for example, can employ polynucleotide terminal transferase to add the desired moiety, such as for example, cordycepin, ³⁵S-dATP, and biotinylated dUTP.

Oligonucleotide derivatives are also available as labels. For example, etheno-dA and etheno-A are known fluorescent adenine nucleotides which can be incorporated into a reporter. Similarly, etheno-dC is another analog that can be used in reporter synthesis. The reporters containing such nucleotide derivatives can be hydrolyzed to release much more strongly fluorescent mononucleotides by the polymerase's 5′ to 3′ nuclease activity as nucleic acid polymerase extends a primer during PCR.

The label of the reporter can be positioned at any suitable location of the reporter. For example, when the reporter comprises more than one nucleotide, the label can be attached to any suitable nucleotide of the reporter sequence. The label can be positioned at the 5′ terminus of the reporter and separated from the reporter sequence that is complementary to the target nucleic acid by a non-complementary sequence. In this embodiment, the reporter comprises a non-natural base that is complementary to the non-natural base of the amplification product, and a sequence that is noncomplementary to the second region of the second primer, and the label is positioned in the sequence that is noncomplementary to the second region. Further, the label can be indirectly attached to a nucleotide of the reporter, using a suitable spacer or chemical linker.

In another embodiment, the labeled reporter comprises a pair of interactive signal-generating labels effectively positioned on the reporter or on the reporter and a second component of the assay (such as the second oligonucleotide) so as to quench the generation of detectable signal when the interactive signal-generating labels are in sufficiently close proximity to each other. Separation of the interactive signal-generating moieties results in the production of a detectable signal. Examples of such labels include dye/quencher pairs or two dye pairs (where the emission of one dye stimulates emission by the second dye).

In an exemplified embodiment, the interactive signal generating pair comprises a fluorophore and a quencher that can quench the fluorescent emission of the fluorophore, as described herein. For example, a quencher may include dimethylaminoazobenzen aminoexal-3-acryinido (Dabcyl) and the fluorophore may be FAM or HEX. Other fluorophore-quencher pairs have been described in Morrison, Detection of Energy Transfer and Fluorescence Quenching in Nonisotopic Probing, Blotting and Sequencing, Academic Press, 1995.

Alternatively, these interactive signal-generating labels can be used in a detection method where the second region of the second primer comprises at least one non-natural base and a label. The second label of the pair is provided by the reporter, which comprises at least one non-natural base that is complementary to the non-natural base of the second primer, and a second label. For example, if a dye/quencher pair is used, hybridization of the reporter to or incorporation of the amplification product will result in a reduction of fluorescence.

Alternatively, the proximity of the two labels can be detected using fluorescence resonance energy transfer (FRET) or fluorescence polarization. FRET is a distance-dependent interaction between the electronic excited states of two dye molecules in which excitation is transferred from a donor molecule to an acceptor molecule without emission of a photon. Examples of donor/acceptor dye pairs for FRET are known in the art and may include fluorophores and quenchers described herein such as Fluorescein/Tetramethylrhodamine, IAEDANS™/Fluorescein (Molecular Probes, Eugene, Oreg.), EDANS™/Dabcyl, Fluorescein/Fluorescein (Molecular Probes, Eugene, Oreg.), BODIPY™ FL/BODIPY™ FL (Molecular Probes, Eugene, Oreg.), and Fluorescein/QSY7™.

### Annealing of a Reporter Comprising a Non-Natural Base

In one embodiment, the reporter is added following amplification of the target nucleic acid. After PCR has produced sufficient amplification product, the reporter may be annealed to the single stranded region of the amplification product. In this some embodiments, the reporter comprises a dye, a quencher, and a non-natural base that is complementary to the non-natural base of the first and/or second primer. The reporter anneals to the sequence of the first and/or second primer comprising the non-natural base. The reporter can be added to the reaction mix after PCR has produced sufficient amplification product, or the reporter can be added to the reaction mix prior to PCR amplification. Preferably, the reporter is added to the reaction mix prior to PCR amplification. After amplification, the temperature is preferably lowered to a temperature lower than the melting temperature of the reporter/amplification product to allow annealing of the reporter to the single-stranded region of the amplification product. In one embodiment, the reaction temperature is lowered to about 49° C. or less during the step of annealing the reporter to the single-stranded overhang region. Annealing is performed similarly for other embodiments of the methods and kits including those using other reporters and other types of labels, as described above. In another embodiment, the reporter is annealed at or above the melting temperature of the first and second primers and the amplification product.

### Incorporation of a Reporter Comprising Non-Natural Bases

In some embodiments, a region of the first and/or second primer comprises a non-natural base. A non-natural base that is complementary to the non-natural base present in the first and/or second primer is incorporated into the amplification product using a suitable enzyme. In this embodiment, the incorporation of the non-natural base is correlated with the presence of the target nucleic acid in the sample.

The disclosed methods and kits may employ a reporter; a nucleic acid polymerase (not shown); a first primer and a second primer. The PCR reaction mixture may include the four naturally occurring deoxynucleotide triphosphates (i.e., dATP, dCTP, dGTP, and dTTP) as well as one or more non-natural nucleotide triphosphate (or an oligonucleotide containing a non-natural nucleotide triphosphate) as the reporter. In some embodiments, the one or more non-natural nucleotide triphosphates in the reaction mixture comprises a label, which may include a dye and/or a quencher

The first primer may comprise a sequence complementary to a portion of a target nucleic acid and can hybridize to that portion of the target nucleic acid. The second primer may have a first region and a second region. The first region may comprises a sequence complementary to a portion of the target sequence. The second region of the second primer may comprise a sequence that is not complementary to the target nucleic acid and may comprise at least one non-natural base. It will be understood that the second region can include additional nucleotides. In suitable embodiments, the non-natural base is located at the junction between the first region and the second region of the second primer. In some embodiments, the non-natural base present in the second region of the second oligonucleotide primer is an iso-C or an iso-G.

In addition to the first primer and second primer, the sample is reacted or contacted with a polymerase, and a polymerase chain reaction is performed. If the target nucleic acid is present in the sample, the complementary portion of the first primer and the complementary portion of the second primer anneal to the corresponding regions of the target nucleic acid following standard base-pairing rules. When the primers are annealed to the target, the 3′ terminal nucleotide of the first primer is separated from the 3′ terminal nucleotide of the second primer by a sequence of nucleotides, or a “gap.” In a preferred embodiment, the first and second primers are designed such that gap of between about zero (0) to about five (5) bases on the template nucleic acid exists between the 3′ ends of the PCR primers when annealed to the template nucleic acid.

The polymerase is used to synthesize a single strand from the 3′-OH end of each primer using polymerase chain reaction. The polymerase chain reaction is allowed to proceed for the desired number of cycles, to obtain an amplification product.

The reporter may be incorporated into the amplification product opposite the non-natural base. In some embodiments, the non-natural base of the reporter comprises a nucleotide triphosphate base that is complementary to the non-natural base of the single-stranded region of the amplification product. In this embodiment, the PCR reaction includes the presence of labeled non-natural nucleotide triphosphate base, in addition to the four naturally occurring nucleotide triphosphate bases (i.e., dATP, dCTP, dGTP, and dTTP). The concentration of non-natural nucleotide triphosphate base in the PCR reaction can range, for example, from 1 μM to 100 μM. The non-natural nucleotide triphosphate base may include a label.

Suitable enzymes for incorporation of the reporter into the amplification product include, for example, polymerases and ligases. A number of polymerases that are capable of incorporating natural nucleotides into an extending primer chain can also incorporate a non-natural base into an amplification product opposite a complementary non-natural base. Typically, class A DNA polymerases; such as Klenow, Tfl, Tth, Taq, Hot Tub, and Bst, are better able than class B polymerases; such as Pfu, Tli, Vent exo-, T4, and Pwo, to incorporate a non-natural base. Reverse transcriptases, such as HIV-1, can also be used to incorporate non-natural bases into an extending primer opposite its complementary non-natural base within a template. In this embodiment the polymerase can be nuclease deficient or can have reduced nuclease activity. While not intended to limit the disclosed methods and kits, nuclease deficient polymerases are expected to be more robust because nuclease activities have been shown to interfere with some PCR reactions (Gene 1992 112(1):29-35 and Science 1993 260(5109):778-83).

Presence of the target nucleic acid in the sample is determined by correlating the presence of the reporter in the amplification product. Suitable detection and visualization methods are used to detect the target nucleic acid. For example, presence of the target nucleic acid may be determined by detecting the label by fluorescence or other visualization method. Fluorescence polarization, for example, can be used to detect the incorporation of the reporter into the amplification product.

In other embodiments, the reporter comprises a non-natural base (which is complementary to a non-natural base present in the first and/or second primer), and a quencher. In this embodiment, the non-natural base of the first and/or second primer includes a dye. In this embodiment, incorporation of the reporter brings the quencher into proximity with the dye. This, in turn, reduces the signal output of the dye, and this reduction in signal can be detected and correlated with the presence of the target nucleic acid. Suitable dye-quencher pairs are discussed above. Alternatively, a dye-dye pair can be used for fluorescence induction. When the target nucleic acid is present, PCR creates a duplexed product that places the two dyes in close proximity, and the fluorescent output of the label changes. The change is detectable by bench-top fluorescent plate readers or using a real-time PCR detection system.

### Detection

Detection and analysis of the reporter (or oligonucleotide fragments thereof) can be accomplished using any methods known in the art. Numerous methods are available for the detection of nucleic acids containing any of the above-listed labels. For example, biotin-labeled oligonucleotide(s) can be detected using non-isotopic detection methods which employ avidin conjugates such as streptavidin-alkaline phosphatase conjugates. Fluorescein-labeled oligonucleotide(s) can be detected using a fluorescein-imager.

In one embodiment, when the target is present, a duplexed product is created that places the first and second labels (e.g. dye/dye pair) into close proximity. When the two labels are in close proximity, the fluorescent output of the reporter molecule label changes. The change is detectable by most bench-top fluorescent plate readers. Alternatively, the label pair comprises a quencher-label pair in close proximity. In this embodiment, the fluorescent output of the reporter molecule label changes, and this change is detectable. Other suitable detection methods are contemplated for used in the disclosed methods and kits.

In another embodiment, the reporter is detected after further processing. It is contemplated that the reporter oligonucleotide fragments can be separated from the reaction using any of the many techniques known in the art useful for separating oligonucleotides. For example, the reporter oligonucleotide fragments can be separated from the reaction mixture by solid phase extraction. The reporter oligonucleotide fragments can be separated by electrophoresis or by methods other than electrophoresis. For example, biotin-labeled oligonucleotides can be separated from nucleic acid present in the reaction mixture using paramagnetic or magnetic beads, or particles which are coated with avidin (or streptavidin). In this manner, the biotinylated oligonucleotide/avidin-magnetic bead complex can be physically separated from the other components in the mixture by exposing the complexes to a magnetic field. In one embodiment, reporter oligonucleotide fragments are analyzed by mass spectrometry.

In some embodiments, when amplification is performed and detected on an instrument capable of reading fluorescence during thermal cycling, the intended PCR product from non-specific PCR products can be differentiated. Amplification products other than the intended products can be formed when there is a limited amount of template nucleic acid. This can be due to a primer dimer formation where the second primer is incorporated into a primer dimer with itself or the first primer. During primer dimer formation the 3′ ends of the two primers hybridize and are extended by the nucleic acid polymerase to the 5′ end of each primer involved. This creates a substrate that when formed is a perfect substrate for the primers involved to exponentially create more of this non-specific products in subsequent rounds of amplification. Therefore, the initial formation of the primer dimer does not need to be a favorable interaction since even if it is a very rare event the amplification process can allow the dimer product to overwhelm the reaction, particularly when template nucleic acid is limited or absent. When the first and/or second oligonucleotide primer is incorporated into this product a labeled nonstandard base is placed orthogonal to the nonstandard base of the second primer. This results in an interaction between the labels of the reporter and of the first and/or second primer which may give a detectable fluorescent change upon melting. Primer dimer products are typically shorter in length than the intended product and therefore have a lower melting temperature. Since the labels are held in close proximity across the duplex an event that would separate the two strands would disrupt the interaction of the labels. Increasing the temperature of the reaction which contains the reaction products to above the Tₘ of the duplexed DNAs of the primer dimer and intended product may melt the DNA duplex of the product and disrupt the interaction of the labels giving a measurable change in fluorescence. By measuring the change in fluorescence while gradually increasing the temperature of the reaction subsequent to amplification and signal generation it may be possible to determine the Tₘ of the intended product as well as that of the nonspecific product.

The methods may include determining the melting temperature of at least one nucleic acid in a sample (e.g., “amplified nucleic acid” otherwise called “an amplicon”), which may be used to identify the nucleic acid. Determining the melting temperature may include exposing an amplicon to a temperature gradient and observing a detectable signal from a fluorophore. Optionally, where the oligonucleotides of the method are labeled with a first fluorescent dye, determining the melting temperature of the detected nucleic acid may include observing a signal from a second fluorescent dye that is different from the first fluorescent dye. In some embodiments, the second fluorescent dye for determining the melting temperature of the detected nucleic acid is an intercalating agent. Suitable intercalating agents may include, but are not limited to SYBR™ Green 1 dye, SYBR dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

Typically, an intercalating agent used in the method will exhibit a change in fluorescence when intercalated in double-stranded nucleic acid. A change in fluorescence may include an increase in fluorescence intensity or a decrease in fluorescence intensity. For example, the intercalating agent may exhibit a increase in fluorescence when intercalated in double-stranded nucleic acid, and a decrease in fluorescence when the double-stranded nucleic acid is melted. A change in fluorescence may include a shift in fluorescence spectra (i.e., a shift to the left or a shift to the right in maximum absorbance wavelength or maximum emission wavelength). For example, the intercalating agent may emit a fluorescent signal of a first wavelength (e.g., green) when intercalated in double-stranded nucleic and emit a fluorescent signal of a second wavelength (e.g., red) when not intercalated in double-stranded nucleic acid. A change in fluorescence of an intercalating agent may be monitored at a gradient of temperatures to determine the melting temperature of the nucleic acid (where the intercalating agent exhibits a change in fluorescence when the nucleic acid melts).

In the disclosed methods, each of these amplified target nucleic acids may have different melting temperatures. For example, each of these amplified target nucleic acids may have a melting temperature that differs by at least about 1° C., more preferably by at least about 2° C., or even more preferably by at least about 4° C. from the melting temperature of any of the other amplified target nucleic acids.

### Kits

Reagents employed in the disclosed methods can be packaged into diagnostic kits. Diagnostic kits include at least a first and second. In some embodiments the kit includes non-natural bases capable of being incorporated into an elongating oligonucleotide by a polymerase. In one embodiment, the non-natural bases are labeled. If the oligonucleotide and non-natural base are unlabeled, the specific labeling reagents can also be included in the kit. The kit can also contain other suitably packaged reagents and materials needed for amplification, for example, buffers, dNTPs, or polymerizing enzymes, and for detection analysis, for example, enzymes and solid phase extractants.

Reagents useful for the disclosed methods can be stored in solution or can be lyophilized. When lyophilized, some or all of the reagents can be readily stored in microtiter plate wells for easy use after reconstitution. It is contemplated that any method for lyophilizing reagents known in the art would be suitable for preparing dried down reagents useful for the disclosed methods.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EXAMPLES

The present methods and kits, thus generally described, will be understood more readily by reference to the following examples, which are provided by way of illustration and are not intended to be limiting of the present methods and kits.

### Example 1

### Detection of JAK2 Nucleic Acids

A quantitative allele/primer specific real-time PCR assay was developed for detecting the JAK2 V617F mutation. The assay extrapolated a quantitative percentage of JAK2 V617F DNA relative to wt JAK2 DNA with a sensitivity of approximately 1 copy of JAK2 V617F DNA in a background of 1000 wt JAK2 DNA equivalents (i.e., 0.1%).

MultiCode-RTx DNA Reagents were obtained from EraGen Biosciences (2× Isolution, PCR-grade water, 25 mM magnesium chloride, and EraGen PCR buffer). A JAK2 primer mix was obtained from EraGen Biosciences which was designed to produce a 50 bp product upon amplification of JAK2 V617F DNA or wt JAK2 DNA. In this example, the primer mixture included a first specific forward primer for amplifying mutant JAK2 DNA (200 nM) and a second specific forward primer for amplifying wt JAK2 DNA (200 nM). The mixture included a single reverse primer for amplifying both mutant JAK2 DNA and wt JAK2 DNA (i.e., a “universal” reverse primer, 600 nM): Forward Primers: 5′-FAM-XGGTCCTGGTTTTAAATTATGGAGTATGTT (Mutant) (SEQ ID NO:4) and 5′-HEX-XCCAGGAGGTTTTAAATTATGGAGTATGTG (Wild Type) (SEQ ID NO:5); Reverse Primer: 5′-GAACCAGAATATTCTCGTCTCCACAG (Universal Primer) (SEQ ID NO:6), where FAM=fluorescein; HEX=hexachlorofluorescein; and X=isocytosine. The IsoSolution contained iso-G conjugated to a Dabcyl quencher. Reaction conditions were as follows: 95° C., 2 min; two cycles of 95° C., 5 sec; 53° C., 10 sec; 72° C., 20 sec; fifty-five cycles of 95° C., 5 sec; 63° C., 10 sec; 72° C., 20 sec. A positive control and sensitivity standard of 0.1% JAK2 V617F DNA was included in each PCR run.

### Analysis of Concentration Standards-Sensitivity

JAK2 V617F concentration standards consisting of 50%, 25%, 10%, 5%, 1% and 0.1% JAK2 V617F mutant DNA relative to wt JAK2 DNA were created. Source-stock solutions of mutant JAK2 V617F DNA (IVS-044) were obtained from InVivoScribe and wild-type human genomic DNA was obtained from Novagen. Each of the concentration standards was assayed according to the methods described above. The results are shown in Table 6 and indicate that JAK2 V617F mutant DNA was detectable at a concentration of 0.1% relative to wt JAK2 DNA with Ct's ranging from 38.6-43.7 (mean 41.5±1.9) (See Table 6). This indicated a lower limit of detection (LLD) of 1 copy of JAK2 V617F mutant DNA in a background of 1000 wt JAK2 DNA equivalents.

TABLE 6
Sensitivity Studies Using Control Standards

| | | V617F Mutant | | | |
|---|---|---|---|---|---|
| | | Allele | | Wildtype Allele | |
| | Repli- | | Melt Temp | | Melt Temp |
| | cate | Ct | (Het; | Ct | (Het; |
| Control | # | Value | 75.5° C.) | Value | 76.1° C.) |
| 10% Mutant JAK2 | 1 | 30.2 | 75.9 | 26.0 | 77.2 |
| 10% Mutant JAK2 | 2 | 30.2 | 75.7 | 25.9 | 76.9 |
| 10% Mutant JAK2 | 3 | 29.6 | 74.7 | 25.4 | 76.2 |
| 10% Mutant JAK2 | 4 | 29.2 | 76.0 | 26.7 | 77.1 |
| 10% Mutant JAK2 | 5 | 30.0 | 75.9 | 27.3 | 76.7 |
| 10% Mutant JAK2 | 6 | 30.1 | 75.2 | 27.2 | 76.1 |
| 1% Mutant JAK2 | 1 | 33.9 | 75.7 | 25.4 | 77.2 |
| 1% Mutant JAK2 | 2 | 34.3 | 75.6 | 25.1 | 76.9 |
| 1% Mutant JAK2 | 3 | 33.9 | 74.7 | 25.1 | 76.1 |
| 1% Mutant JAK2 | 4 | 34.3 | 76.0 | 26.9 | 77.2 |
| 1% Mutant JAK2 | 5 | 33.5 | 75.4 | 26.4 | 76.6 |
| 1% Mutant JAK2 | 6 | 35.1 | 75.0 | 27.5 | 76.3 |
| 0.1% Mutant JAK2 | 1 | 43.6 | 75.8 | 25.2 | 77.2 |
| 0.1% Mutant JAK2 | 2 | 41.1 | 75.1 | 25.2 | 76.8 |
| 0.1% Mutant JAK2 | 3 | 38.6 | 75.0 | 25.1 | 76.1 |
| 0.1% Mutant JAK2 | 4 | 41.7 | 75.9 | 26.8 | 76.6 |
| 0.1% Mutant JAK2 | 5 | 43.7 | 75.6 | 27.2 | 76.6 |
| 0.1% Mutant JAK2 | 6 | 40.5 | 75.2 | 27.1 | 76.3 |
| 0.01% Mutant JAK2 | 1 | N/A | N/A | 25.1 | 76.8 |
| 0.01% Mutant JAK2 | 2 | N/A | N/A | 25.1 | 76.8 |
| 0.01% Mutant JAK2 | 3 | N/A | N/A | 25.1 | 75.9 |
| 0.01% Mutant JAK2 | 4 | N/A | N/A | 27.3 | 77.1 |
| 0.01% Mutant JAK2 | 5 | N/A | N/A | 27.0 | 76.5 |
| 0.01% Mutant JAK2 | 6 | N/A | N/A | 27.2 | 76.3 |
| 0% Mutant JAK2 | 1 | N/A | N/A | 25.3 | 76.9 |
| 0% Mutant JAK2 | 2 | N/A | N/A | 25.1 | 76.7 |
| 0% Mutant JAK2 | 3 | N/A | N/A | 24.8 | 76.6 |
| 0% Mutant JAK2 | 4 | N/A | N/A | 27.3 | 76.7 |
| 0% Mutant JAK2 | 5 | N/A | N/A | 27.1 | 76.5 |
| 0% Mutant JAK2 | 6 | N/A | N/A | 27.6 | 76.3 |

### Reproducibility Studies—Concentration Standards

A triplicate assessment of concentration standards (50%, 25%, 10%, 5%, 1% JAK2 V617F mutant DNA) was performed on two Light Cycler instruments over a period of 4 days. The mean ΔCt of 24 replicates on Light Cyclers 1 and 2 are shown in Table 7.

TABLE 7
Reproducibility Studies - Concentration Standards

| | | | | | | |
|---|---|---|---|---|---|---|
| | | Wild | | | | |
| | Mutant | type | Delta | % | Coef- | Con- |
| Sample ID | Ct | Ct | Ct | Mutant | ficient | stant |

Light Cycler #1

| | | | | | | |
|---|---|---|---|---|---|---|
| 50% Mutant Control | 27.02 | 26.83 | 0.19 | 49.26 | −2.2249 | 8.8622 |
| 25% Mutant Control | 28.23 | 26.49 | 1.74 | 24.54 | | |
| 10% Mutant Control | 30.22 | 26.27 | 3.95 | 9.10 | | |
| 5% Mutant Control | 31.11 | 26.29 | 4.82 | 6.16 | | |
| 1% Mutant Control | 35.07 | 26.03 | 9.04 | 0.92 | | |

Light Cycler #2

| | | | | | | |
|---|---|---|---|---|---|---|
| 50% Mutant Control | 26.89 | 26.82 | 0.08 | 48.66 | −2.1893 | 8.5802 |
| 25% Mutant Control | 28.12 | 26.59 | 1.53 | 25.09 | | |
| 10% Mutant Control | 30.01 | 26.29 | 3.72 | 9.22 | | |
| 5% Mutant Control | 31.19 | 26.52 | 4.68 | 5.95 | | |
| 1% Mutant Control | 35.10 | 26.37 | 8.73 | 0.93 | | |

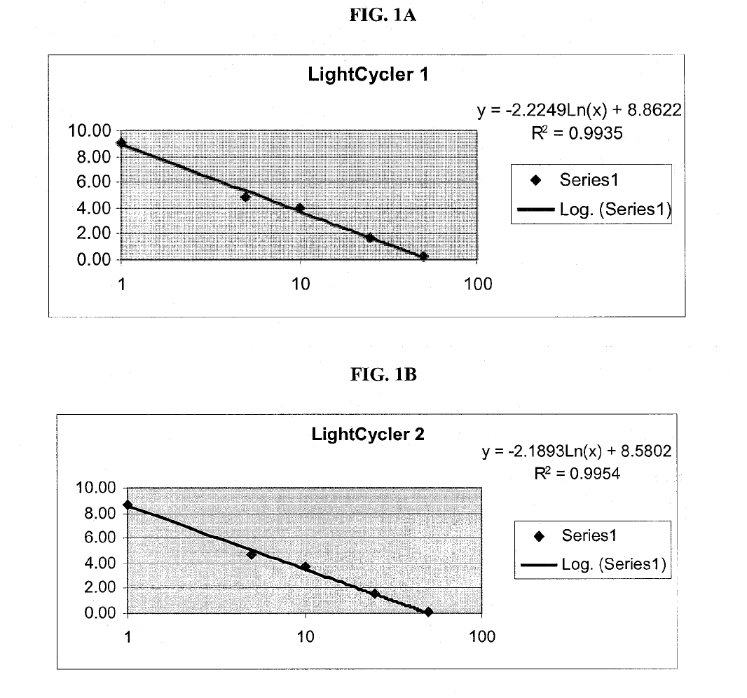

Comparisons among groups of concentration standards for each analysis (day) show no significant differences (p≧0.05) in the ΔCt's observed. Comparison of all results for each concentration standard between the two Light Cyclers indicated no significant differences in the performance of this assay using the two instruments (p≧0.05). Plotting the logs of the ΔCt for each concentration showed expected linearity on both instruments (FIGS. 1A and 1B). As such, the assay shows highly reproducible results across instruments.

### Determination of Performance Characteristics at LLD of 0.1%

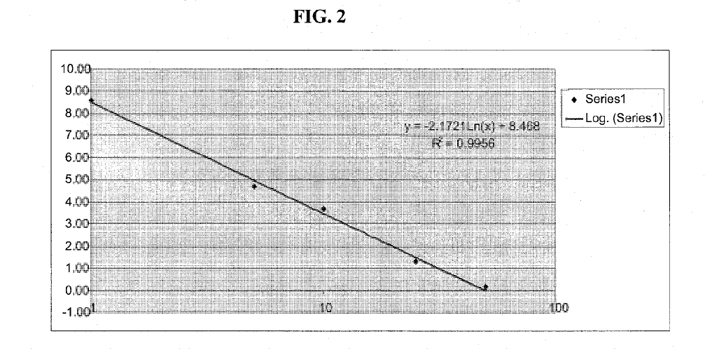

The logs of the ΔCt's from 24 replicates of each concentration standard were utilized to generate a standard curve and corresponding linear equation (See Table 8, FIG. 2).

TABLE 8
Analysis of Concentration Standards

| | | Wild | | | | |
|---|---|---|---|---|---|---|
| | Mutant | type | Delta | % | Coef- | Con- |
| Sample ID | Ct | Ct | Ct | Mutant | ficient | stant |
| 50% Mutant Control | 26.95 | 26.82 | 0.13 | 46.39 | −2.1721 | 8.468 |
| 25% Mutant Control | 28.18 | 26.90 | 1.28 | 27.43 | | |
| 10% Mutant Control | 30.11 | 26.45 | 3.66 | 9.14 | | |
| 5% Mutant Control | 31.15 | 26.45 | 4.70 | 5.67 | | |
| 1% Mutant Control | 35.08 | 26.50 | 8.58 | 0.95 | | |

This “master” standard curve was then utilized to determine the precision and accuracy of JAK2 V617F mutation detection at the LLD of 0.1%. Using the master standard curve and corresponding equation, percentage mutant values were extrapolated for 24 replicates of the JAK-2 V617F 0.1% DNA concentration standards (Table 9). Samples were tested on two different instruments (LC #1 or #2) over four days by two different operators. The extrapolated percentages (N=24) ranged from 0.02-0.27 with a mean of 0.08 (±0.06). The performance characteristics of the assay is summarized in Table 10. The assay shows highly reproducible results across instruments and operators.

TABLE 9
Extrapolation of JAK2 Mutant DNA (%) Relative to Wild-type

| | % V617 F | | | |
|---|---|---|---|---|
| Rep # | 50-1% STD | LC# | Day # | Operator |
| 1 | 0.05 | 1 | 1 | A |
| 2 | 0.04 | | | |
| 3 | 0.07 | | | |
| 4 | 0.03 | 2 | | |
| 5 | 0.04 | | | |
| 6 | 0.08 | | | |
| 7 | 0.12 | 1 | 2 | B |
| 8 | 0.16 | | | |
| 9 | 0.03 | | | |
| 10 | 0.03 | 2 | | |
| 11 | 0.15 | | | |
| 12 | 0.04 | | | |
| 13 | 0.27 | 1 | 3 | A |
| 14 | 0.02 | | | |
| 15 | 0.05 | | | |
| 16 | 0.16 | 2 | | |
| 17 | 0.03 | | | |
| 18 | 0.05 | | | |
| 19 | 0.04 | 1 | 4 | B |
| 20 | 0.08 | | | |
| 21 | 0.04 | | | |
| 22 | 0.09 | 2 | | |
| 23 | 0.06 | | | |
| 24 | 0.07 | | | |
| Mean | 0.08 | | | |
| STD-DEV | 0.06 | | | |
| MIN | 0.02 | | | |
| MAX | 0.27 | | | |

TABLE 10
Performance Characteristics of JAK-2
V617F Mutation Detection Assay

| LLD | 0.1% V617F Mutant DNA |
|---|---|
| Sensitivity | 1 copy of V617F in a |
| | background of 1000 genome |
| | equivalents |
| Specificity | Approaches 100% |
| Accuracy of Extrapolated Value | 80% |
| Precision of Extrapolated Value | 75% |

### Patient Specimens Analyzed

The assay was used to analyze 527 blood and bone marrow specimens. The specimens were obtained from patients who had been referred for JAK-2 V617F mutation analysis based on suspicious morphology and/or flow cytometry. The assay indicated that 149 (28%) specimens were positive and 378 (72%) were negative. As such, these results demonstrate that the methods of the present invention are useful in the analysis of JAK-2 mutations for clinical specimens.

### Example 2

### Comparison of Alternate Primer Designs

In this Example, a comparison was made between designs of reverse allele-specific primers and forward allele-specific primers. MultiCode-RTx DNA Reagents were obtained from EraGen Biosciences (2× Isolution, PCR-grade Water, 25 mM Magnesium Chloride, and EraGen PCR buffer). Reaction conditions were as follows: 95° C., 2 min; two cycles of 95° C., 5 sec; 53° C., 10 sec; 72° C., 20 sec; fifty-five cycles of 95° C., 5 sec; 63° C., 10 sec; 72° C., 20 sec. All assays were run on an ABI 7900 or ABI 7700 Sequence Detection System.

Six reverse ASP systems were tested with all seven common forward primers on a set of synthetic JAK targets (WT, Mutant, WT/Mutant Mix, NTC). Three forward systems were tested with all common reverse primers. The forward system included the RUO designs (SEQ ID NOS: 4, 5, and 6) as described in Example 1. The JAK2 synthetic targets were tested at varying concentrations of mutant DNA (0%, 1%, 10%, 50%, 90%, 99%, and 100% mutant DNA).

TABLE 11
JAK2 Primer Combinations

| System | Wild Type ASP | Mutant ASP | Universal Primer |
|---|---|---|---|
| JAK2 F1 | SEQ ID NO: 39 | SEQ ID NO: 45 | SEQ ID NO: 51 |
| | | | SEQ ID NO: 52 |
| | | | SEQ ID NO: 53 |
| | | | SEQ ID NO: 54 |
| | | | SEQ ID NO: 55 |
| | | | SEQ ID NO: 6 |
| JAK2 F2 | SEQ ID NO: 42 | SEQ ID NO: 48 | SEQ ID NO: 51 |
| | | | SEQ ID NO: 52 |
| | | | SEQ ID NO: 53 |
| | | | SEQ ID NO: 54 |
| | | | SEQ ID NO: 55 |
| | | | SEQ ID NO: 6 |
| JAK RUO | SEQ ID NO: 5 | SEQ ID NO: 4 | SEQ ID NO: 51 |
| Forward | | | SEQ ID NO: 52 |
| | | | SEQ ID NO: 53 |
| | | | SEQ ID NO: 54 |
| | | | SEQ ID NO: 55 |
| | | | SEQ ID NO: 6 |
| JAK2 R1 | SEQ ID NO: 15 | SEQ ID NO: 27 | SEQ ID NO: 8 |
| | | | SEQ ID NO: 9 |
| | | | SEQ ID NO: 10 |
| | | | SEQ ID NO: 11 |
| | | | SEQ ID NO: 12 |
| | | | SEQ ID NO: 13 |
| | | | SEQ ID NO: 14 |
| JAK2 R2 | SEQ ID NO: 18 | SEQ ID NO: 65 | SEQ ID NO: 8 |
| | | | SEQ ID NO: 9 |
| | | | SEQ ID NO: 10 |
| | | | SEQ ID NO: 11 |
| | | | SEQ ID NO: 12 |
| | | | SEQ ID NO: 13 |
| | | | SEQ ID NO: 14 |
| JAK2 R3 | SEQ ID NO: 21 | SEQ ID NO: 33 | SEQ ID NO: 8 |
| | | | SEQ ID NO: 9 |
| | | | SEQ ID NO: 10 |
| | | | SEQ ID NO: 11 |
| | | | SEQ ID NO: 12 |
| | | | SEQ ID NO: 13 |
| | | | SEQ ID NO: 14 |
| JAK2 R4 | SEQ ID NO: 21 | SEQ ID NO: 36 | SEQ ID NO: 8 |
| | | | SEQ ID NO: 9 |
| | | | SEQ ID NO: 10 |
| | | | SEQ ID NO: 11 |
| | | | SEQ ID NO: 12 |
| | | | SEQ ID NO: 13 |
| | | | SEQ ID NO: 14 |

Wild type primer concentration was tested at both 200 nM and 150 nM. Mutant primer concentration was 200 nM. Common reverse primers were tested at 400 nM and 600 nM concentrations.

In a preliminary experiment, each primer system in Table 11, including one wild-type primer, one mutant primer, and a common reverse primer was tested for sensitivity and lack of primer dimer formation. It was shown that the JAK2 R4 reverse ASP primer system worked well in the detection assays with either forward primer DM1172 (SEQ ID NO:12), DM1169 (SEQ ID NO:9), or DM1170 (SEQ ID NO: 10). These primer combinations yielded very few primer dimers.

It was shown that the JAK2 F2 forward ASP primer system worked well in detection assays with all common reverse primers (SEQ ID NOS:51-55), including the original RUO common reverse primer (SEQ ID NO: 6). The penultimate mismatch system (JAK2 F1) functioned well with five of the six common reverse primers (SEQ ID NO: 51, 52, 53, 55, 6) demonstrating sensitivity down to a 1% mutant in wildtype mixture and showed very few primer dimers. The original RUO system (Fwd WT primer: SEQ ID NO: 5; Fwd Mut Primer: SEQ ID NO: 4) worked well with two of the six common reverse (SEQ ID NO: 53, 6) demonstrating sensitivity down to a 1% mutant in wildtype mixture and showed very few primer dimers. For the JAK2 F1 assay, optimal primer concentrations were determined to be WT primer (SEQ ID NO: 39): 130 nM; Mutant Primer (SEQ ID NO: 45): 200 nM; and common reverse primer (SEQ ID NO: 52): 600 nM.

### Feasibility Study

The ASP F1 system [WT primer (SEQ ID NO: 39): 130 nM; Mutant Primer (SEQ ID NO: 45): 200 nM; and common reverse primer (SEQ ID NO: 52): 600 nM], the system described in Example 1, and an optimized conditions of Example 1 [WT primer (SEQ ID NO: 5): 200 nM; Mutant Primer (SEQ ID NO: 4): 250 nM; and common reverse primer (SEQ ID NO: 6): 600 nM] were evaluated at varying concentrations of mutant DNA (0%, 0.01%, 0.1%, 1%, 5%, 10%, 25%, 50%, 75%, 90%, and 100% mutant DNA).

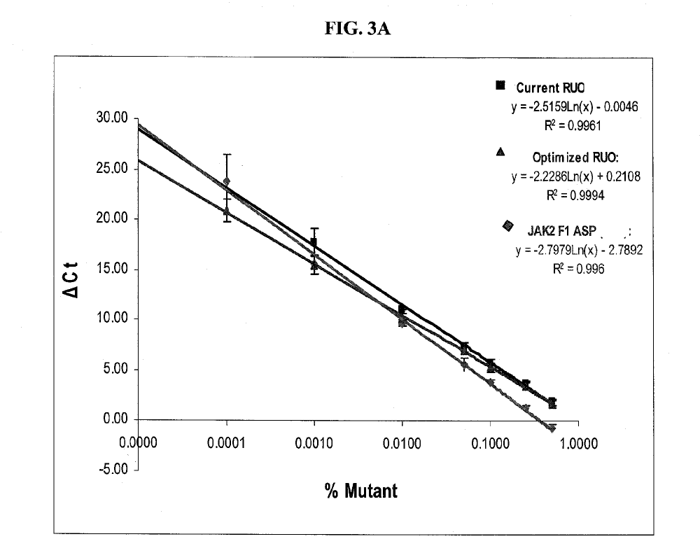

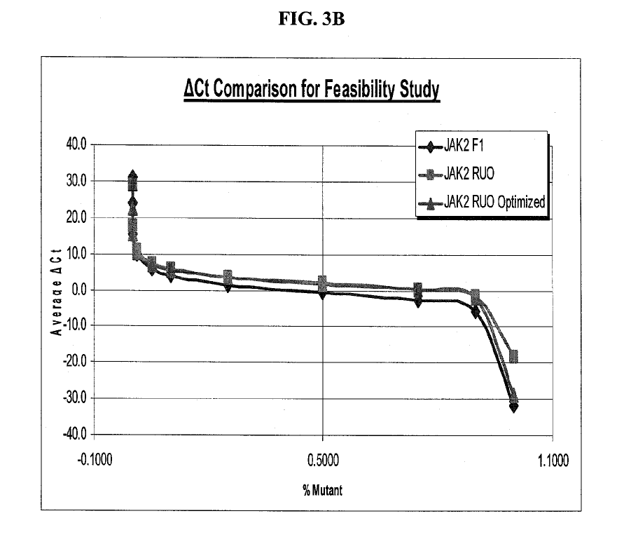

Briefly, the ASP F1 system demonstrated a sensitivity of 0.01% mutant in a wild-type mixture in 8/8 replicates and no primer dimers were detected (Table 14). The optimized RUO condition demonstrated a sensitivity of 0.01% mutant in a wild-type mixture in 5/8 replicates and no primer dimers were detected (Table 13). The RUO conditions demonstrated a sensitivity of 0.1% mutant in a wild-type mixture in 8/8 replicates and several primer dimers were detected (Table 12). At the 50% mutant level, a suitable detection method would have Ct values for the mutant and wild-type channels approximately equal yielding a ΔCt value near zero. The ASP F1 system matched these conditions most closely (Table 15) and had the most symmetrical plot though the dilution series (FIG. 3B).

TABLE 12
ΔCt Comparison - RUO

| | | | | | | NS (outside | |
|---|---|---|---|---|---|---|---|
| Percent | Average Ct | | Average | Average Tm | | of Tm Call) | |
| Mutant | WT | Mutant | ΔCt | WT | Mutant | WT | Mutant |
| 0.00% | 24.6 ± 0.3 | 41.2 | 28.7 ± 4.9 | 74.7 ± 0.1 | 74.4 | N/A | N/A |
| | (8/8) | (1/8) | | (8/8) | (1/8) | | |
| 0.01% | 24.9 ± 0.4 | N/A | N/A | 74.8 ± 0.1 | N/A | N/A | Ct |
| | (8/8) | | | (8/8) | | | 51.5 ± 2.1, |
| | | | | | | | Tm |
| | | | | | | | 71.9 ± 0.1 |
| | | | | | | | (3/8) |
| 0.10% | 25.1 ± 0.4 | 42.9 ± 1.5 | 17.8 ± 1.3 | 74.7 ± 0.2 | 72.8 ± 0.2 | N/A | N/A |
| | (8/8) | (8/8) | | (8/8) | (8/8) | | |
| 1.00% | 25.1 ± 0.3 | 36.1 ± 0.4 | 11.0 ± 0.3 | 74.7 ± 0.2 | 73.3 ± 0.2 | N/A | N/A |
| | (8/8) | (8/8) | | (8/8) | (8/8) | | |
| 5.00% | 25.5 ± 0.4 | 32.7 ± 0.4 | 7.3 ± 0.4 | 74.6 ± 0.2 | 73.6 ± 0.2 | N/A | N/A |
| | (8/8) | (8/8) | | (8/8) | (8/8) | | |
| 10.00% | 25.4 ± 0.4 | 31.1 ± 0.3 | 5.8 ± 0.3 | 74.7 ± 0.1 | 73.7 ± 0.1 | N/A | N/A |
| | (8/8) | (8/8) | | (8/8) | (8/8) | | |
| 25.00% | 25.7 ± 0.4 | 29.4 ± 0.3 | 3.7 ± 0.3 | 74.6 ± 0.1 | 73.8 ± 0.1 | N/A | N/A |
| | (8/8) | (8/8) | | (8/8) | (8/8) | | |
| 50.00% | 26.3 ± 0.4 | 28.1 ± 0.2 | 1.9 ± 0.3 | 74.5 ± 0.2 | 73.8 ± 0.1 | N/A | N/A |
| | (8/8) | (8/8) | | (8/8) | (8/8) | | |
| 75.00% | 27.3 ± 0.3 | 27.4 ± 0.3 | 0.1 ± 0.2 | 74.3 ± 0.2 | 73.8 ± 0.2 | N/A | N/A |
| | (8/8) | (8/8) | | (8/8) | (8/8) | | |
| 90.00% | 28.6 ± 0.4 | 27.1 ± 0.2 | −1.6 ± 0.4 | 74.1 ± 0.2 | 73.8 ± 0.2 | N/A | N/A |
| | (8/8) | (8/8) | | (8/8) | (8/8) | | |
| 100.00% | 44.5 ± 2.4 | 26.9 ± 0.3 | 18.4 ± 4.4 | 74.2 ± 0.6 | 73.8 ± 0.1 | N/A | N/A |
| | (8/8) | (8/8) | | (8/8) | (8/8) | | |
| NTC | 39.0 ± 0.7 | 47.0 | N/A | 75.1 ± 0.2 | 74.0 (1/8) | N/A | Ct 51.1, |
| | (8/8) | (1/8) | | (8/8) | | | Tm 75.5 |
| | | | | | | | (1/8) |

TABLE 13
ΔCt Comparison - Optimized RUO

| | | | | | | NS (outside | |
|---|---|---|---|---|---|---|---|
| Percent | Average Ct | | Average | Average Tm | | of Tm Call) | |
| Mutant | WT | Mutant | ΔCt | WT | Mutant | WT | Mutant |
| 0.00% | 24.6 ± 0.2 | 45.2 | 29.2 ± 3.3 | 74.3 ± 0.1 | 73.2 | N/A | N/A |
| | (8/8) | (1/8) | | (8/8) | (1/8) | | |
| 0.01% | 24.4 ± 0.2 | 45.4 ± 1.2 | 20.9 ± 1.1 | 74.3 ± 0.1 | 73.0 ± 0.2 | N/A | N/A |
| | (8/8) | (5/8) | | (8/8) | (5/8) | | |
| 0.10% | 24.6 ± 0.2 | 40.1 ± 0.9 | 15.5 ± 0.9 | 74.3 ± 0.2 | 73.2 ± 0.2 | N/A | N/A |
| | (8/8) | (8/8) | | (8/8) | (8/8) | | |
| 1.00% | 24.7 ± 0.3 | 34.9 ± 0.7 | 10.2 ± 0.8 | 74.2 ± 0.1 | 73.3 ± 0.2 | N/A | N/A |
| | (8/8) | (8/8) | | (8/8) | (8/8) | | |
| 5.00% | 24.8 ± 0.3 | 31.8 ± 0.4 | 7.0 ± 0.4 | 74.2 ± 0.1 | 73.5 ± 0.1 | N/A | N/A |
| | (8/8) | (8/8) | | (8/8) | (8/8) | | |
| 10.00% | 24.9 ± 0.2 | 30.3 ± 0.3 | 5.3 ± 0.4 | 74.2 ± 0.1 | 73.5 ± 0.1 | N/A | N/A |
| | (8/8) | (8/8) | | (8/8) | (8/8) | | |
| 25.00% | 25.2 ± 0.2 | 28.6 ± 0.4 | 3.5 ± 0.4 | 74.2 ± 0.1 | 73.5 ± 0.1 | N/A | N/A |
| | (8/8) | (8/8) | | (8/8) | (8/8) | | |
| 50.00% | 25.6 ± 0.3 | 27.3 ± 0.2 | 1.7 ± 0.4 | 74.0 ± 0.1 | 73.4 ± 0.1 | N/A | N/A |
| | (8/8) | (8/8) | | (8/8) | (8/8) | | |
| 75.00% | 26.6 ± 0.3 | 26.6 ± 0.3 | 0.0 ± 0.4 | 73.9 ± 0.2 | 73.3 ± 0.1 | N/A | N/A |
| | (8/8) | (8/8) | | (8/8) | (8/8) | | |
| 90.00% | 28.3 ± 0.3 | 26.3 ± 0.3 | −2.0 ± 0.5 | 73.8 ± 0.1 | 73.3 ± 0.1 | N/A | N/A |
| | (8/8) | (8/8) | | (8/8) | (8/8) | | |
| 100.00% | N/A | 26.2 ± 0.3 | 28.8 ± 0.3 | N/A | 73.3 ± 0.1 | N/A | N/A |
| | | (8/8) | | | (8/8) | | |
| NTC | 41.1 | N/A | N/A | 74.4 | N/A | N/A | N/A |
| | (1/8) | | | (1/8) | | | |

TABLE 14
ΔCt Comparison - ASP F1

| | | | | | | NS (outside | |
|---|---|---|---|---|---|---|---|
| Percent | Average Ct | | Average | Average Tm | | of Tm Call) | |
| Mutant | WT | Mutant | ΔCt | WT | Mutant | WT | Mutant |
| 0.00% | 24.0 ± 0.1 | N/A | 31.0 ± 0.1 | 75.0 ± 0.1 | N/A | N/A | N/A |
| | (8/8) | | | (8/8) | | | |
| 0.01% | 23.9 ± 0.2 | 47.7 ± 2.5 | 23.8 ± 2.6 | 75.0 ± 0.1 | 74.3 ± 0.1 | N/A | N/A |
| | (8/8) | (8/8) | | (8/8) | (8/8) | | |
| 0.10% | 23.9 ± 0.3 | 39.5 ± 0.7 | 15.6 ± 0.7 | 75.0 ± 0.1 | 74.4 ± 0.1 | N/A | N/A |
| | (8/8) | (8/8) | | (8/8) | (8/8) | | |
| 1.00% | 24.0 ± 0.2 | 33.6 ± 0.3 | 9.7 ± 0.3 | 74.9 ± 0.1 | 74.3 ± 0.1 | N/A | N/A |
| | (8/8) | (8/8) | | (8/8) | (8/8) | | |
| 5.00% | 24.3 ± 0.4 | 29.9 ± 0.8 | 5.6 ± 0.6 | 74.9 ± 0.1 | 74.4 ± 0.1 | N/A | N/A |
| | (8/8) | (8/8) | | (8/8) | (8/8) | | |
| 10.00% | 24.5 ± 0.4 | 28.2 ± 0.4 | 3.8 ± 0.3 | 74.9 ± 0.1 | 74.4 ± 0.1 | N/A | N/A |
| | (8/8) | (8/8) | | (8/8) | (8/8) | | |
| 25.00% | 24.8 ± 0.2 | 26.0 ± 0.4 | 1.3 ± 0.3 | 74.8 ± 0.1 | 74.4 ± 0.1 | N/A | N/A |
| | (8/8) | (8/8) | | (8/8) | (8/8) | | |
| 50.00% | 25.4 ± 0.3 | 24.7 ± 0.3 | −0.7 ± 0.4 | 74.7 ± 0.1 | 74.3 ± 0.1 | N/A | N/A |
| | (8/8) | (8/8) | | (8/8) | (8/8) | | |
| 75.00% | 26.9 ± 0.4 | 24.1 ± 0.2 | −2.8 ± 0.5 | 74.6 ± 0.1 | 74.3 ± 0.1 | N/A | N/A |
| | (8/8) | (8/8) | | (8/8) | (8/8) | | |
| 90.00% | 29.2 ± 0.4 | 23.5 ± 0.4 | −5.7 ± 0.5 | 74.5 ± 0.1 | 74.2 ± 0.1 | N/A | N/A |
| | (8/8) | (8/8) | | (8/8) | (8/8) | | |
| 100.0% | N/A | 23.4 ± 0.4 | 31.7 ± 0.4 | N/A | 74.3 ± 0.1 | N/A | N/A |
| | | (8/8) | | | (8/8) | | |
| NTC | 39.7 | N/A | N/A | 74.4 | N/A | N/A | N/A |
| | (1/8) | | | (1/8) | | | |

TABLE 15
ΔCt Comparison

| | 0.00% | 0.01% | 0.10% | 1.00% | 5.00% | 10.00% | 25.00% | 50.00% | 75.00% | 90.00% | 100.00% |
|---|---|---|---|---|---|---|---|---|---|---|---|
| JAK2 F1 | 31.0 | 23.8 | 15.6 | 9.7 | 5.6 | 3.8 | 1.3 | −0.7 | −2.8 | −5.7 | −31.7 |
| JAK2 RUO | 28.7 | N/A | 17.8 | 11.0 | 7.3 | 5.8 | 3.7 | 1.9 | 0.1 | −1.6 | −18.4 |
| JAK2 RUO Optimized | 29.2 | 20.9 | 15.5 | 10.2 | 7.0 | 5.3 | 3.5 | 1.7 | 0.0 | −2.0 | −28.8 |

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member, any subgroup of members of the Markush group or other group, or the totality of members of the Markush group or other group.

Also, unless indicated to the contrary, where various numerical values are provided for embodiments, additional embodiments are described by taking any 2 different values as the endpoints of a range. Such ranges are also within the scope of the described invention.

What is claimed is:

1. A method of detecting wt JAK2 nucleic acid and mutant JAK2 nucleic acid in a sample, if present, the method comprising:
(a) contacting the sample with:
(i) a first primer suitable for amplifying a wt JAK2 nucleic acid, wherein the first primer comprises a first label and a first non-natural base;
(ii) a second primer suitable for amplifying a mutant JAK2 nucleic acid, wherein the second primer comprises a second label and a second occurrence of the first non-natural base;
(iii) a third primer suitable for amplifying both wt JAK2 nucleic acid and mutant JAK2 nucleic acid; and
(iv) a reporter comprising a third label and a second non-natural base that base-pairs with the first non-natural base;
(b) performing an amplification reaction comprising the primers of step (a) under conditions suitable to produce an amplification product of the wt JAK2 nucleic acid and the mutant JAK2 nucleic acid in the sample, if present, wherein the reporter is incorporated into the amplification products; and
(c) detecting the amplification products produced in step (b) by observing a signal from the first label, the second label, or both, thereby determining the presence or absence of wt JAK2 nucleic acid, mutant JAK2 nucleic acid, or both in the sample.

2. The method of claim 1, wherein the first label comprises a first fluorophore and the second label comprises a second fluorophore.

3. The method of claim 2, wherein the first fluorophore is one of FAM or HEX and the second fluorophore is the other of FAM or HEX.

4. The method of claim 1, wherein the third label comprises a quencher.

5. The method of claim 4, wherein the quencher is Dabcyl.

6. The method of claim 1, wherein the signal from the first label, the second label, or both is observed during the reaction.

7. The method of claim 6, wherein the signal from the first label decreases during the reaction when the wt JAK2 nucleic acid is present in the sample and the signal from the second label decreases during the reaction when the mutant JAK2 nucleic acid is present in the sample.

8. The method of claim 1, wherein the step of detecting the amplification products comprises measuring the amount of signal from the first label, the second label, or both during the reaction thereby quantifying the relative amount of wt JAK2 nucleic acid and the mutant JAK2 nucleic acid in the sample.

9. The method of claim 1, further comprising the step of determining the melting temperature of the amplification product of the wt JAK2 nucleic acid and the mutant JAK2 nucleic acid in the sample, if present, wherein the signal from the first label, the second label, or both, increases upon melting of the amplification products.

10. The method of claim 1, wherein the first non-natural base is iso-C or iso-G and the second non-natural base is the other of iso-C or iso-G.

11. The method of claim 1, wherein the first primer, the second primer, or both the first primer and the second primer comprise a 5′ tail, wherein the 5′ tail comprises from 5 to 10 nucleotides that are non-complementary to JAK2 nucleic acid.

12. The method of claim 1, wherein the first primer comprises a sequence selected from the group consisting of: SEQ ID NOS: 5, 15-26, 39-44, 56-57, and complements thereof.

13. The method of claim 1, wherein the second primer comprises a sequence selected from the group consisting of SEQ ID NOS: 4, 27-38, 45-50, 58-59 and complements thereof.

14. The method of claim 1, wherein the third primer comprises a sequence selected from the group consisting of: SEQ ID NOS:6-14, 51-55, and complements thereof.

15. The method of claim 1, wherein the first primer comprises SEQ ID NO: 5, the second primer comprises SEQ ID NO: 4, and the third primer comprises SEQ ID NO: 6.

16. The method of claim 1, wherein the first primer comprises SEQ ID NO:39, the second primer comprises SEQ ID NO:45, and the third primer comprises SEQ ID NO:52.

17. The method of claim 1, wherein the first primer comprises SEQ ID NO:21, the second primer comprises SEQ ID NO:36, and the third primer is selected from the group consisting of: SEQ ID NO: 9, 10, and 12.

18. The method of claim 1, wherein the sample comprises no more than about 1% mutant JAK2 nucleic acid relative to wt JAK2 nucleic acid.

19. The method of claim 1, wherein the sample comprises no more than about 0.1% mutant JAK2 nucleic acid relative to wt JAK2 nucleic acid.

20. The method of claim 1, wherein the second primer is complementary to mutant JAK2 V617F nucleic acid.
